(19) United States
(12) Patent Application Publication
(10) Pub. No.: US 2023/0031305 A1
(43) Pub. Date: Feb. 2, 2023

(54) COMPOSITIONS AND METHODS FOR ANALYSIS USING NUCLEIC ACID PROBES AND BLOCKING SEQUENCES

(71) Applicant: 10x Genomics, Inc., Pleasanton, CA (US)

(72) Inventors: Jorge Iván HERNÁNDEZ NEUTA, Stockholm (SE); Toon VERHEYEN, Solna (SE)

(21) Appl. No.: 17/877,664

(22) Filed: Jul. 29, 2022

Related U.S. Application Data
(60) Provisional application No. 63/227,824, filed on Jul. 30, 2021.

(51) Int. Cl. C12Q 1/6832; C12Q 1/6841; C12Q 1/682

(52) U.S. Cl. CPC C12Q 1/6832; C12Q 1/6841; C12Q 1/682

(57) ABSTRACT

The present disclosure relates in some aspects to methods for analyzing a target nucleic acid in a biological sample. In some aspects, provided herein are methods and compositions for detecting a region of interest in a target nucleic acid, wherein hybridization between an interrogatory region of a probe and a region of interest of the target nucleic acid is blocked by a blocking strand unless the interrogatory region is complementary to the region of interest. In some aspects, the methods provided herein increase specificity of detecting a region of interest in a target nucleic acid (e.g., a SNP in an RNA molecule). In some aspects, the presence, amount, and/or identity of a region of interest in a target nucleic acid is analyzed in situ. Also provided are polynucleotides, sets of polynucleotides, compositions, and kits for use in accordance with the methods, for example for RNA-targeting padlock probe-mediated SNP detection.

### CROSS-REFERENCE TO RELATED APPLICATIONS

This application claims priority to U.S. Provisional Patent Application No. 63/227,824, filed Jul. 30, 2021, entitled “COMPOSITIONS AND METHODS FOR ANALYSIS USING NUCLEIC ACID PROBES AND BLOCKING SEQUENCES,” which is herein incorporated by reference in its entirety for all purposes.

### FIELD

The present disclosure relates in some aspects to methods and compositions for analysis of a target nucleic acid, such as the detection of a nucleic acid sequence of interest in situ in a biological sample or in an array of biomolecules.

### BACKGROUND

Methods are available for analyzing nucleic acids present in a biological sample, such as a cell or a tissue. For instance, advances in single molecule fluorescent in situ hybridization (smFISH) have enabled nanoscale-resolution imaging of RNA in cells and tissues. However, analysis of short sequences (e.g., single nucleotide polymorphisms (SNPs) or point mutations) on individual transcripts has remained challenging, and often suffers from low assay specificity and/or stringency and high rates of false positive results. Improved methods for analyzing nucleic acids present in a biological sample with increased specificity and stringency are needed. Provided herein are methods and compositions that address such and other needs.

### BRIEF SUMMARY

Provided herein generally are methods and compositions for analyzing a region of interest (e.g., a short sequence of interest such as a single nucleotide of interest) of a target nucleic acid. In some embodiments, provided herein is a method for detecting a region of interest in a target nucleic acid, the method comprising: a) providing i) a probe comprising an interrogatory region and ii) a blocking strand, wherein the blocking strand is hybridized to a hybridization region in the probe or the target nucleic acid, thereby blocking the hybridization region from hybridizing to a complementary hybridization region in the target nucleic acid or the probe, respectively, and the blocking strand comprises a blocking sequence complementary to the interrogatory region in the probe or the region of interest in the target nucleic acid; b) allowing hybridization between the probe and the target nucleic acid, wherein if the interrogatory region is complementary to the region of interest, the blocking strand is displaced and the hybridization region is available for hybridizing to the target nucleic acid or the probe; c) ligating the probe hybridized to the target nucleic acid to itself or to another probe hybridized to the target nucleic acid; and d) detecting the ligated probe or an amplification product thereof, thereby detecting the region of interest in the target nucleic acid.

In some embodiments, a blocking strand disclosed herein may be bound to a probe prior to contacting a target nucleic acid with the probe and/or the blocking strand. In some embodiments, provided herein is a method for detecting a region of interest in a target nucleic acid, the method comprising: a) providing i) a probe comprising an interrogatory region and ii) a blocking strand, wherein the blocking strand is hybridized to a hybridization region in the probe, thereby blocking the hybridization region from hybridizing to a complementary hybridization region in the target nucleic acid, and the blocking strand comprises a blocking sequence complementary to the interrogatory region in the probe; b) allowing hybridization between the probe and the target nucleic acid, wherein if the interrogatory region is complementary to the region of interest, the blocking strand is displaced and the hybridization region is available for hybridizing to the target nucleic acid; c) ligating the probe hybridized to the target nucleic acid to itself or to another probe hybridized to the target nucleic acid; and d) detecting the ligated probe or an amplification product thereof, thereby detecting the region of interest in the target nucleic acid.

In some embodiments, a blocking strand disclosed herein may be bound to a target nucleic acid prior to contacting a probe with the target nucleic acid and/or the blocking strand. In some embodiments, provided herein is a method for detecting a region of interest in a target nucleic acid, the method comprising: a) providing i) a probe comprising an interrogatory region and ii) a blocking strand, wherein the blocking strand is hybridized to a hybridization region in the target nucleic acid, thereby blocking the hybridization region from hybridizing to a complementary hybridization region in the probe, and the blocking strand comprises a blocking sequence complementary to the region of interest in the target nucleic acid; b) allowing hybridization between the probe and the target nucleic acid, wherein if the interrogatory region is complementary to the region of interest, the blocking strand is displaced and the hybridization region is available for hybridizing to the probe; c) ligating the probe hybridized to the target nucleic acid to itself or to another probe hybridized to the target nucleic acid; and d) detecting the ligated probe or an amplification product thereof, thereby detecting the region of interest in the target nucleic acid.

In any of the preceding embodiments, the blocking strand and the probe can be in different molecules. In some embodiments, the probe is a circularizable probe and the blocking strand is not part of the circularizable probe.

Alternatively, in any of the preceding embodiments, the blocking strand and the probe can be in the same molecule. In some embodiments, the probe is a circularizable probe and the blocking strand is part of the circularizable probe. In some embodiments, the blocking strand and the hybridization region of the probe form a duplex prior to the hybridization between the probe and the target nucleic acid in step b). In some embodiments, the blocking sequence and the interrogatory region form a duplex in the circularizable probe prior to the hybridization between the hybridization region and the target nucleic acid in step b).

In any of the preceding embodiments, the probe can be a circularizable probe (e.g., a padlock probe), where the ligating in step c) circularizes the circularizable probe. In any of the preceding embodiments, the circularizable probe can be circularized using the target nucleic acid as a template, with or without gap filling prior to ligation. In any of the preceding embodiments, the circularization may comprise ligating the circularizable probe using the target nucleic acid as a template, with or without gap filling prior to ligation. In any of the preceding embodiments, the circularization may comprise ligating the circularizable probe using a splint as a template, with or without gap filling prior to ligation. In some embodiments, the circularizable probe can be circularized using a splint as a template, where the splint and the target nucleic acid are not the same molecule. In some embodiments, the splint is not hybridized to the target nucleic acid. In some embodiments, the splint comprises a sequence capable of hybridizing to the target nucleic acid. In some embodiments, the splint is hybridized to the target nucleic acid. In some embodiments, the target nucleic acid is RNA and the circularizable probe and/or the splint are DNA or primarily DNA. The circularizable probe and/or the splint may each comprise a DNA backbone and one or more ribonucleotides. In any of the preceding embodiments, the method can comprise generating a rolling circle amplification (RCA) product of the circularized circularizable probe and detecting the RCA product. The RCA product may be generated and detected in situ in a biological sample such as a tissue section or an array of biomolecules.

In any of the preceding embodiments, the probe can be a first probe and the ligating in step c) can comprise ligating the first probe to a second probe hybridized to the target nucleic acid. The first and second probes may hybridize to adjacent regions in the target nucleic acid. In some embodiments, the first and second probes are ligated using the target nucleic acid as a template, with or without gap filling prior to ligation. In some embodiments, the first and second probes are ligated using a splint as a template, where the splint and the target nucleic acid are not the same molecule. In some embodiments, the target nucleic acid is RNA and the splint is DNA or primarily DNA. In some embodiments, the splint comprises a DNA backbone and one or more ribonucleotides. In any of the preceding embodiments, the method can comprise detecting a ligation product of the first and second probes.

In any of the preceding embodiments, the target nucleic acid can comprise RNA. In any of the preceding embodiments, the target nucleic acid can comprise DNA. In any of the preceding embodiments, the target nucleic acid can comprise DNA residue(s) and/or RNA residue(s). In any of the preceding embodiments, the target nucleic acid can be an mRNA. In any of the preceding embodiments, the blocking strand can comprise DNA residue(s) and/or non-DNA residue(s), such as RNA or PNA. In any of the preceding embodiments, the probe can comprise DNA residue(s) and/or RNA residue(s). In any of the preceding embodiments, the probe is a chimeric DNA-RNA probe and can comprise primarily DNA and one or more ribonucleotides, e.g., no more than four consecutive ribonucleotides. In some embodiments, the one or more ribonucleotides are at and/or near a ligatable 3′ end of the probe. In any of the preceding embodiments, the probe can comprise a ribonucleotide at its 3′ end. In some embodiments, the probe is a padlock probe comprising a ribonucleotide which is optionally at the 3′ end of the padlock probe.

In any of the preceding embodiments, the interrogatory region can be an internal region in the probe. In some embodiments, the interrogatory region is not at the 3′ or 5′ end of the probe. In some embodiments, the interrogatory region does not comprise a 3′ or 5′ terminal nucleotide of the probe. In any of the preceding embodiments, the interrogatory region can be one, two, three, four, five, six, seven, eight, nine, ten, or more nucleotides from the 3′ or 5′ terminal nucleotide of the probe. In any of the preceding embodiments, the interrogatory region can be one, two, three, four, or five nucleotides in length. In any of the preceding embodiments, the interrogatory region can be a single nucleotide.

In any of the preceding embodiments, the blocking sequence can be at the 3′ or 5′ end of the blocking strand or between the 3′ and 5′ ends of the blocking strand. In some embodiments, the blocking sequence can comprise a 3′ terminal nucleotide. In some embodiments, the blocking sequence can comprise a 5′ terminal nucleotide. In any of the preceding embodiments, the blocking sequence can be one, two, three, four, or five nucleotides in length. In any of the preceding embodiments, the blocking sequence can be a single nucleotide.

In any of the preceding embodiments, the region of interest can be one, two, three, four, or five nucleotides in length. In any of the preceding embodiments, the region of interest can be a single nucleotide. In any of the preceding embodiments, the interrogatory region, the blocking sequence, and the region of interest can each be a single nucleotide.

In some embodiments, the single nucleotide interrogatory region and the single nucleotide blocking sequence are complementary to each other, and a hybridization complex preformed between the probe and the blocking strand is contacted with the target nucleic acid. In some embodiments, the single nucleotide region of interest is complementary to the single nucleotide interrogatory region. In some embodiments, hybridization of a sequence (e.g., a toehold region) of the probe to the target nucleic acid initiates strand displacement, and the single nucleotide blocking sequence and the single nucleotide region of interest compete with each other for hybridization to the single nucleotide interrogatory region. In some embodiments, basepairing between the single nucleotide region of interest and the single nucleotide interrogatory region is favored, such that branch migration proceeds to displace the single nucleotide blocking sequence from the probe. In some embodiments, the blocking strand is displaced from the probe.

In some embodiments, the single nucleotide region of interest and the single nucleotide blocking sequence are complementary to each other, and a hybridization complex preformed between the target nucleic acid and the blocking strand is contacted with the probe. In some embodiments, the single nucleotide region of interest is complementary to the single nucleotide interrogatory region. In some embodiments, hybridization of a sequence (e.g., a toehold region) of the target nucleic acid to the probe initiates strand displacement, and the single nucleotide blocking sequence and the single nucleotide interrogatory region compete with each other for hybridization to the single nucleotide region of interest. In some embodiments, basepairing between the single nucleotide region of interest and the single nucleotide interrogatory region is favored, such that branch migration proceeds to displace the single nucleotide blocking sequence from the target nucleic acid. In some embodiments, the blocking strand is displaced from the target nucleic acid.

In any of the preceding embodiments, the region of interest can be selected from the group consisting of a single-nucleotide polymorphism (SNP), a single-nucleotide variant (SNV), a single-nucleotide substitution, a point mutation, a single-nucleotide deletion, and a single-nucleotide insertion.

In any of the preceding embodiments, the hybridization region can be between about 5 and about 50 nucleotides in length. In any of the preceding embodiments, the hybridization region can be between about 5 and about 10, between about 10 and about 15, or between about 15 and about 20 nucleotides in length.

In some embodiments, the interrogatory region is in the hybridization region of the probe, the region of interest is in the complementary hybridization region of the target nucleic acid, and the hybridization region hybridizes to the complementary hybridization region. In some embodiments, the blocking strand hybridizes to the interrogatory region in the hybridization region of the probe. The interrogatory region can be at the 5′ end, at the 3′ end, or between the 5′ end and the 3′ end of the hybridization region of the probe.

In any of the preceding embodiments, the probe can comprise a toehold region adjacent to the interrogatory region. In any of the preceding embodiments, the target nucleic acid can comprise a toehold region adjacent to the region of interest. In any of the preceding embodiments, the toehold region can hybridize to the target nucleic acid or the probe, thereby allowing displacement of the blocking strand from the probe or the target nucleic acid, respectively. In any of the preceding embodiments, the toehold region can be between about 5 and about 50 nucleotides in length. In any of the preceding embodiments, the toehold region can be between about 5 and about 10, between about 10 and about 15, or between about 15 and about 20 nucleotides in length. In any of the preceding embodiments, the toehold region can be at the 3′ or 5′ end of the probe. In any of the preceding embodiments, the toehold region can be at or near the 3′ end of a padlock probe. In some embodiments, the toehold region is in a padlock probe and comprises a 3′ terminal nucleotide. In any of the preceding embodiments, the toehold region can be at or near the 5′ end of a padlock probe. In some embodiments, the toehold region is in a padlock probe and comprises a 5′ terminal nucleotide.

In any of the preceding embodiments, in the ligating of step c), the probe can be hybridized to the target nucleic acid via the hybridization region, for instance, after the blocking strand or a portion thereof is displaced from the hybridization region to render it available for hybridization to the target nucleic acid or the probe, where the interrogatory region in the probe hybridizes to the region of interest in the target nucleic acid. In some embodiments, in the ligating of step c), the probe is more stably hybridized to the target nucleic acid compared to when the blocking strand or a portion thereof is not displaced from the probe or the target nucleic acid. In some embodiments, in the ligating of step c), the probe is more stably hybridized to the target nucleic acid compared to when the hybridization region or a portion thereof is blocked by the blocking strand.

In any of the preceding embodiments, the ligation can be selected from the group consisting of enzymatic ligation, chemical ligation, template dependent ligation, and/or template independent ligation. In any of the preceding embodiments, the enzymatic ligation can comprise using a ligase having a DNA-templated ligase activity. In any of the preceding embodiments, the enzymatic ligation can comprise using a ligase having an RNA-templated ligase activity. In any of the preceding embodiments, the enzymatic ligation can comprise using a ligase having an RNA-templated DNA ligase activity and/or an RNA-templated RNA ligase activity. In any of the preceding embodiments, the enzymatic ligation can comprise using a ligase selected from the group consisting of a Chlorella virus DNA ligase (PB CV DNA ligase), a T4 RNA ligase, a T4 DNA ligase, and a single-stranded DNA (ssDNA) ligase. In any of the preceding embodiments, the enzymatic ligation can comprise using a PBCV-1 DNA ligase or variant or derivative thereof and/or a T4 RNA ligase 2 (T4 Rnl2) or variant or derivative thereof.

In any of the preceding embodiments, the method can further comprise prior to the ligating step, a step of removing probe molecules that are not bound to the target nucleic acid. In any of the preceding embodiments, the method can further comprise prior to the ligating step, a step of removing probe molecules that are bound to the target nucleic acid but comprise in the interrogatory region one or more mismatches with the region of interest, and/or allowing probe molecules or portions thereof comprising one or more mismatches to dissociate from the target nucleic acid while probe molecules comprising no mismatch in the interrogatory region remain bound to the target nucleic acid. In some embodiments, under the same conditions, probe molecules comprising one or more mismatches can be less stably bound to the target nucleic acid than probe molecules comprising no mismatch in the interrogatory region. In some embodiments, the removing step and/or the allowing step comprise one or more stringency washes.

In some embodiments, a complex between a probe and a blocking strand is formed prior to contacting the complex with a target nucleic acid. In these embodiments, under the same conditions, duplex formation and/or duplex stability between the blocking strand and probe molecules comprising the one or more mismatches (with the region of interest) in the interrogatory region are favored, whereas duplex formation and/or duplex stability between the blocking strand and probe molecules comprising no mismatch (with the region of interest) are disfavored. For probes comprising the one or more mismatches, after contacting with the target nucleic acid, displacement of the blocking strand from the probe by the target nucleic acid is disfavored. In some of these embodiments, after contacting with the target nucleic acid, displacement of the blocking strand from the probe by the target nucleic acid is not initiated. In some of these embodiments, after contacting with the target nucleic acid, the one or more mismatches (with the region of interest) in the interrogatory region prevent and/or stall branch migration such that hybridization and/or rehybridization of the blocking strand to the probe is favored. In some of these embodiments, under the same conditions, duplex formation and/or duplex stability between the target nucleic acid and probe molecules comprising the one or more mismatches are disfavored, whereas duplex formation and/or duplex stability between the target nucleic acid and probe molecules comprising no mismatch in the interrogatory region are favored. For probes comprising no mismatches, after contacting with the target nucleic acid, displacement of the blocking strand from the probe by the target nucleic acid is initiated. In some of these embodiments, after contacting with the target nucleic acid, displacement of the blocking strand from the probe by the target nucleic acid is favored. In some of these embodiments, after contacting with the target nucleic acid, the complementarity between the interrogatory region and the region of interest favors hybridization and/or rehybridization of the target nucleic acid to the probe, such that branch migration proceeds in a direction to displace the blocking strand or a portion thereof.

In some embodiments, a complex between a target nucleic acid and a blocking strand is formed prior to contacting the complex with a probe. In these embodiments, under the same conditions, duplex formation and/or duplex stability between the blocking strand and probe molecules comprising the one or more mismatches (with the region of interest) in the interrogatory region are disfavored, whereas duplex formation and/or duplex stability between the blocking strand and probe molecules comprising no mismatch (with the region of interest) are favored. For probes comprising the one or more mismatches, after contacting with the target nucleic acid, displacement of the blocking strand from the target nucleic acid by the probe is disfavored. In some of these embodiments, after contacting with the target nucleic acid, displacement of the blocking strand from the target nucleic acid by the probe is not initiated. In some of these embodiments, after contacting with the target nucleic acid, the one or more mismatches (with the region of interest) in the interrogatory region prevent and/or stall branch migration such that hybridization and/or rehybridization of the blocking strand to the target nucleic acid is favored. In some of these embodiments, under the same conditions, duplex formation and/or duplex stability between the target nucleic acid and probe molecules comprising the one or more mismatches are disfavored, whereas duplex formation and/or duplex stability between the target nucleic acid and probe molecules comprising no mismatch in the interrogatory region are favored. For probes comprising no mismatches, after contacting with the target nucleic acid, displacement of the blocking strand from the target nucleic acid by the probe is initiated. In some of these embodiments, after contacting with the target nucleic acid, displacement of the blocking strand from the target nucleic acid by the probe is favored. In some of these embodiments, after contacting with the target nucleic acid, the complementarity between the interrogatory region and the region of interest favors hybridization and/or rehybridization of the target nucleic acid to the probe, such that branch migration proceeds in a direction to displace the blocking strand or a portion thereof.

In any of the preceding embodiments, the removing step and/or the allowing step may comprise one or more stringency washes.

In any of the preceding embodiments, the target nucleic acid can be in a biological sample. In any of the preceding embodiments, the ligated probe and/or the amplification product thereof can be generated in situ in the biological sample. In any of the preceding embodiments, the amplification product can be generated using a linear rolling circle amplification (RCA), a branched RCA, a dendritic RCA, or any combination thereof. In any of the preceding embodiments, the amplification product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In any of the preceding embodiments, the ligated probe and/or the amplification product thereof can be immobilized in the biological sample and/or crosslinked to one or more other molecules in the biological sample.

In any of the preceding embodiments, the method can comprise imaging the biological sample to detect the ligated probe and/or the amplification product thereof. In any of the preceding embodiments, the imaging can comprise detecting a signal associated with the ligated probe and/or the amplification product thereof. In any of the preceding embodiments, the signal can be amplified in situ in the biological sample. In some embodiments, the signal amplification in situ can comprises rolling circle amplification (RCA) of a probe that directly or indirectly binds to the ligated probe and/or the amplification product thereof hybridization chain reaction (HCR) directly or indirectly on the ligated probe and/or the amplification product thereof; linear oligonucleotide hybridization chain reaction (LO-HCR) directly or indirectly on the ligated probe and/or the amplification product thereof primer exchange reaction (PER) directly or indirectly on the ligated probe and/or the amplification product thereof assembly of branched structures directly or indirectly on the ligated probe and/or the amplification product thereof; hybridization of a plurality of detectable probes directly or indirectly on the ligated probe and/or the amplification product thereof, or any combination thereof.

In any of the preceding embodiments, the ligated probe and/or the amplification product thereof can be analyzed by sequential hybridization, sequencing by hybridization, sequencing by ligation, sequencing by synthesis, sequencing by binding, or a combination thereof.

In any of the preceding embodiments, the ligated probe and/or the amplification product thereof can comprise one or more barcode sequences or complements thereof. In some embodiments, the one or more barcode sequences or complements thereof correspond to the target nucleic acid and/or the region of interest.

In any of the preceding embodiments, the target nucleic acid can be a viral DNA, bacterial DNA, or cellular DNA or RNA molecule or a product thereof in the biological sample. In any of the preceding embodiments, the target nucleic acid can be genomic DNA, mitochondrial DNA, mRNA or cDNA. In any of the preceding embodiments, the target nucleic acid can be a reporter oligonucleotide of a labelling agent comprising (i) a binding moiety that that directly or indirectly binds to a nucleic acid or non-nucleic acid target molecule and (ii) the reporter oligonucleotide which corresponds to the binding moiety and/or the nucleic acid or non-nucleic acid target molecule.

In any of the preceding embodiments, the one or more barcode sequences or complements thereof can be detected by: contacting the biological sample with one or more detectably-labeled probes that directly or indirectly hybridize to the one or more barcode sequences or complements thereof; detecting signals associated with the one or more detectably-labeled probes; and dehybridizing the one or more detectably-labeled probes. In some embodiments, the contacting, detecting, and dehybridizing steps are repeated with the one or more detectably-labeled probes and/or one or more other detectably-labeled probes that directly or indirectly hybridize to the one or more barcode sequences or complements thereof.

In any of the preceding embodiments, the one or more barcode sequences or complements thereof can be detected by: contacting the biological sample with one or more intermediate probes that directly or indirectly hybridize to the one or more barcode sequences or complements thereof, wherein the one or more intermediate probes are detectable using one or more detectably-labeled probes; detecting signals associated with the one or more detectably-labeled probes; and dehybridizing the one or more intermediate probes and/or the one or more detectably-labeled probes. In some embodiments, the contacting, detecting, and dehybridizing steps are repeated with the one or more intermediate probes, the one or more detectably-labeled probes, one or more other intermediate probes, and/or one or more other detectably-labeled probes.

In some embodiments, disclosed herein is a method for analyzing a target nucleic acid in a biological sample, the method comprising: a) contacting the biological sample with a probe and a blocking strand, wherein: the blocking strand is hybridized to a hybridization region in the probe or the target nucleic acid, thereby blocking the hybridization region from hybridizing to a complementary hybridization region in the target nucleic acid or the probe, respectively, and the probe or the target nucleic acid comprises a toehold region adjacent to the hybridization region; b) allowing hybridization of the toehold region to the target nucleic acid or the probe, whereby the blocking strand is displaced and the hybridization region is available for hybridizing to the target nucleic acid or the probe; c) ligating the probe hybridized to the target nucleic acid via the hybridization region to itself or to another probe hybridized to the target nucleic acid; and d) detecting the ligated probe or an amplification product thereof in the biological sample.

In some embodiments, disclosed herein is a method for detecting a region of interest in a target nucleic acid in a biological sample, the method comprising: a) contacting the target nucleic acid comprising the region of interest with a circularizable probe comprising a metastable stem-loop structure, wherein: a strand of the stem of the metastable stem-loop structure comprises an interrogatory region, and the circularizable probe further comprises a toehold region adjacent to the interrogatory region; b) hybridizing the toehold region to the target nucleic acid, wherein if the interrogatory region is complementary to the region of interest, the other strand of the stem is displaced and the strand comprising the interrogatory region is available for hybridizing to the target nucleic acid; c) circularizing the circularizable probe hybridized to the target nucleic acid via the available strand of the stem; and d) detecting a rolling circle amplification product of the circularized circularizable probe, thereby detecting the region of interest in the target nucleic acid.

In some embodiments, disclosed herein is a method for analyzing a biological sample comprising a plurality of target RNA molecules comprising a single nucleotide of interest, the method comprising: a) contacting the biological sample with a circularizable probe hybridized to a blocking strand, wherein: the blocking strand is hybridized to a hybridization region in the circularizable probe, thereby blocking the hybridization region from hybridizing to a complementary hybridization region in a target RNA molecule, the hybridization region in the circularizable probe comprises an interrogatory nucleotide hybridized to a blocking nucleotide in the blocking strand, and the circularizable probe comprises a toehold region directly linked to the interrogatory nucleotide via a phosphodiester bond; b) allowing hybridization of the toehold region to target RNA molecules in the biological sample, wherein: for a first target RNA molecule in which the single nucleotide of interest is complementary to the interrogatory nucleotide, the blocking strand is displaced and the hybridization region of the circularizable probe is available for hybridizing to the first target RNA molecule, and for a second target RNA molecule in which the single nucleotide of interest is not complementary to the interrogatory nucleotide, the blocking strand is not displaced and the hybridization region of the circularizable probe remains unavailable for hybridizing to the second target RNA molecule; c) allowing molecule(s) of the circularizable probe to dissociate from the second target RNA molecule, under conditions in which molecule(s) of the circularizable probe remain hybridized to the first target RNA molecule; d) circularizing the circularizable probe hybridized to the first target RNA molecule; and e) detecting a rolling circle amplification product of the circularized circularizable probe in the biological sample.

In some embodiments, the circularizable probe is circularized using the first target RNA molecule as a template, with or without gap filling prior to ligation. In some embodiments, the circularizable probe is circularized using a splint as a template. In some embodiments, the splint comprises a sequence hybridized to the first target RNA molecule.

In any of the preceding embodiments, the method can further comprise washing the biological sample to remove molecule(s) of the circularizable probe from the second target RNA molecule, under conditions in which molecule(s) of the circularizable probe remain hybridized to the first target RNA molecule in biological sample. In any of the preceding embodiments, the circularizable probe can comprise 5′ and 3′ arms that hybridize to the target RNA molecules. In some embodiments, the 5′ and 3′ arms are of the same length. In some embodiments, the 5′ and 3′ arms are of different lengths. In some embodiments, the interrogatory nucleotide is in the shorter of the 5′ and 3′ arms. In some embodiments, the interrogatory nucleotide is in the longer of the 5′ and 3′ arms.

In any of the preceding embodiments, the circularizable probe can comprise one or more ribonucleotides. In any of the preceding embodiments, the circularizable probe can comprise no more than four consecutive ribonucleotides. In any of the preceding embodiments, the circularizable probe can comprise a ribonucleotide at its 3′ end.

In some embodiments, disclosed herein is a method for analyzing a target nucleic acid in a biological sample, the method comprising contacting the biological sample with a probe and a blocking strand, wherein the blocking strand is hybridized to a hybridization region in the probe or the target nucleic acid, thereby blocking the hybridization region from hybridizing to a complementary hybridization region in the target nucleic acid or the probe, respectively. In some embodiments, the probe or the target nucleic acid comprises a toehold region adjacent to the hybridization region. In any of the preceding embodiments, the method can further comprise allowing hybridization of the toehold region to the target nucleic acid or the probe, whereby the blocking strand is displaced and the hybridization region is available for hybridizing to the target nucleic acid or the probe. In any of the preceding embodiments, the method can further comprise ligating the probe hybridized to the target nucleic acid via the hybridization region to itself or to another probe hybridized to the target nucleic acid. In any of the preceding embodiments, the method can further comprise detecting the ligated probe or an amplification product thereof in the biological sample.

In some embodiments, disclosed herein is a method for detecting a region of interest in a target nucleic acid in a biological sample, the method comprising contacting the target nucleic acid comprising the region of interest with a circularizable probe comprising a metastable stem-loop structure, wherein a strand of the stem of the metastable stem-loop structure comprises an interrogatory region. In some embodiments, the circularizable probe further comprises a toehold region adjacent to the interrogatory region. In any of the preceding embodiments, the method can further comprise hybridizing the toehold region to the target nucleic acid. In any of the preceding embodiments, the interrogatory region may be complementary to the region of interest, where the other strand of the stem is displaced and the strand comprising the interrogatory region is made available for hybridizing to the target nucleic acid. In any of the preceding embodiments, the method can further comprise circularizing the circularizable probe hybridized to the target nucleic acid via the available strand of the stem. In any of the preceding embodiments, the method can further comprise detecting a rolling circle amplification product of the circularized circularizable probe, thereby detecting the region of interest in the target nucleic acid.

In some embodiments, disclosed herein is a method for analyzing a biological sample comprising a plurality of target RNA molecules comprising a single nucleotide of interest, the method comprising contacting the biological sample with a circularizable probe comprising a metastable stem-loop structure, wherein a strand of the stem of the metastable stem-loop structure comprises an interrogatory nucleotide. In some embodiments, the circularizable probe further comprises a toehold region adjacent to the interrogatory nucleotide. In any of the preceding embodiments, the method can further comprise hybridizing the toehold region to target RNA molecules in the biological sample, wherein: for a first target RNA molecule in which the single nucleotide of interest is complementary to the interrogatory nucleotide, the other strand of the stem is displaced and the strand comprising the interrogatory nucleotide is available for hybridizing to the first target RNA molecule; and for a second target RNA molecule in which the single nucleotide of interest is not complementary to the interrogatory nucleotide, the other strand of the stem is not displaced and the strand comprising the interrogatory nucleotide is unavailable for hybridizing to the second target RNA molecule. In any of the preceding embodiments, the method can further comprise allowing molecule(s) of the circularizable probe to dissociate from the second target RNA molecule, under conditions in which molecule(s) of the circularizable probe remain hybridized to the first target RNA molecule. In any of the preceding embodiments, the method can further comprise circularizing the circularizable probe hybridized to the first target RNA molecule. In any of the preceding embodiments, the method can further comprise detecting a rolling circle amplification product of the circularized circularizable probe in the biological sample.

In any of the preceding embodiments, the circularizable probe can be circularized using the first target RNA molecule as a template, with or without gap filling prior to ligation. In any of the preceding embodiments, the circularizable probe can be circularized using a splint as a template. In some embodiments, the splint comprises a sequence hybridized to the first target RNA molecule.

In any of the preceding embodiments, the method can further comprise washing the biological sample to remove molecule(s) of the circularizable probe from the second target RNA molecule, under conditions in which molecule(s) of the circularizable probe remain hybridized to the first target RNA molecule in biological sample.

In any of the preceding embodiments, the circularizable probe can comprise 5′ and 3′ arms that hybridize to the target RNA molecules. In some embodiments, the 5′ and 3′ arms are of the same length. In some embodiments, the 5′ and 3′ arms are of different lengths.

In any of the preceding embodiments, the circularizable probe can comprise one or more ribonucleotides. In any of the preceding embodiments, the circularizable probe can comprise no more than four consecutive ribonucleotides. In any of the preceding embodiments, the circularizable probe can comprise a ribonucleotide at its 3′ end.

In any of the preceding embodiments, the biological sample can comprise cells or cellular components. In any of the preceding embodiments, the biological sample can be a tissue sample. In any of the preceding embodiments, the biological sample can be fixed. Alternatively, in any of the preceding embodiments, the biological sample may not be fixed. In any of the preceding embodiments, the biological sample can be permeabilized. In any of the preceding embodiments, the biological sample can be a fixed and/or permeabilized biological sample. In any of the preceding embodiments, the biological sample can be a formalin-fixed, paraffin-embedded (FFPE) sample, a frozen tissue sample, or a fresh tissue sample. In any of the preceding embodiments, the biological sample can be a tissue slice between about 1 μm and about 50 μm in thickness, optionally wherein the tissue slice is between about 5 μm and about 35 μm in thickness. In any of the preceding embodiments, the biological sample can be processed. In any of the preceding embodiments, the biological sample can be cleared. In any of the preceding embodiments, the biological sample can be embedded in a matrix. In any of the preceding embodiments, the biological sample can be embedded in a hydrogel. In any of the preceding embodiments, the biological sample can be embedded in a hydrogel and then cleared. In any of the preceding embodiments, the biological sample and/or the matrix can be crosslinked. In any of the preceding embodiments, the biological sample can be immobilized on a substrate, such as a glass or plastic slide.

In some aspects, provided herein is a kit for analyzing a biological sample, comprising a circularizable probe and a blocking strand, wherein the circularizable probe comprises an interrogatory region and a hybridization region and a toehold region flanking the interrogatory region. In some embodiments, the blocking strand is capable of hybridizing to the hybridization region in the circularizable probe, thereby blocking the hybridization region from hybridizing to a complementary hybridization region in a target nucleic acid in the biological sample. In any of the preceding embodiments, the blocking strand can comprise a blocking sequence complementary to the interrogatory region in the circularizable probe. In any of the preceding embodiments, the toehold region can hybridize to the target nucleic acid such that the target nucleic acid displaces the blocking strand from the hybridization region in the circularizable probe. In any of the preceding embodiments, the toehold region can be at the 3′ or 5′ end of the circularizable probe.

In some aspects, provided herein is a kit for analyzing a biological sample, comprising a circularizable probe comprising a metastable stem-loop structure. In some embodiments, a strand of the stem of the metastable stem-loop structure comprises an interrogatory region. In any of the preceding embodiments, the circularizable probe can further comprise a toehold region adjacent to the interrogatory region. In any of the preceding embodiments, the toehold region can hybridize to the target nucleic acid such that the target nucleic acid displaces the other strand of the stem from the interrogatory region in the circularizable probe.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner.

FIG. 1 depicts an exemplary circularizable probe hybridized to a blocking strand, wherein the blocking strand and the probe are in different molecules. The circularizable probe can be a padlock probe comprising a 5′ phosphate group and a 3′ base which is optionally an RNA base, e.g., in cases where the target nucleic acid is an RNA such as an mRNA.

FIG. 2 depicts an exemplary hybridization complex comprising a circularizable probe, a blocking strand, and a target nucleic acid.

FIG. 3 depicts an exemplary circularizable probe comprising a blocking stand, such that hybridization of the blocking strand to the hybridization region results in the formation of a metastable stem-loop structure.

FIG. 4 depicts an exemplary circularizable probe that comprises a metastable stem-loop structure comprising a blocking strand, wherein the blocking strand and the probe are in the same molecule.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In situ methods for detecting mutations or single nucleotide polymorphisms (SNPs) for the purposes of genotyping have typically been performed using fluorescent in situ hybridization (FISH). This is typically low-throughput and limited to cell lines. Higher-throughput approaches, such as, e.g., padlock-based detection of SNPs combined with rolling circle amplification (RCA), can allow for highly specific SNP detection. Such padlock-RCA based SNP detection can use a ligase such as T4 DNA ligase to ligate the padlock probe into a circular molecule, which will only occur when there has been specific base-pairing between the probe and the SNP of interest in the target nucleic acid. However, currently available designs for padlock-based SNP detection combined with RCA suffer from several drawbacks. For example, certain designs require that the base on the probe that is complementary to the SNP of interest be situated precisely on the 3′ end of the padlock ligation site, limiting design options. Additionally, ligation-based nucleic acid detection methods in some cases exhibit poor performance on RNA, e.g., due to reduced fidelity of ligases using an RNA template. Therefore, if the target nucleic acid is, for example, an mRNA, cDNA synthesis may be required to convert the target mRNA into cDNA in order to allow hybridization with the padlock probe. In some cases, requiring cDNA synthesis for RNA targets is detrimental because cDNA synthesis has been shown to lower the sensitivity of padlock-RCA based in situ analysis. In some cases, using padlock probes to directly target RNA without an intervening cDNA step is also problematic, because such methods may not reliably and specifically detect SNPs. Current chimeric padlock methods still suffer from low reliability and specificity for SNP detection due to reduced ligase discrimination capacity at the ligation site.

Certain ligation-based assays to identify and/or distinguish between sequence(s) of interest in a target nucleic acid can suffer from lack of specificity for multiple reasons, including properties of the ligase and/or the target nucleic acid. This lack of fidelity can result in formation and detection as a ligation product, even when the sequence of interest does not match the interrogatory region of a probe, producing a high level of background or false positive results. For instance, the SplintR® Ligase (also known as the PBCV-1 DNA Ligase or Chlorella virus DNA Ligase) efficiently catalyzes RNA-templated ligation of DNA ends. However, the ligase may not discriminate sequences while it ligates and can have high template independent ligation efficiency. For example, a conventional padlock probe having an interrogatory base G may be designed to detect the complementary C of a SNP (e.g., in an RNA transcript). When the ligase fidelity is low, however, the padlock probe may be ligated when hybridized to a target having an A, T/U, or G in the SNP position, leading to false positive results. Using additional padlock probes having interrogatory bases other than G may not increase assay specificity; in fact, the additional “control” padlock probes may themselves suffer from template independent ligation and consequently false positive results. Thus, there is a need for ligation methods with higher specificity for detection of short sequences (e.g., one or more nucleotides) such as SNP genotyping in situ.

The present disclosure provides methods and compositions for analysis of regions of interest in a target nucleic acid, such as a single nucleotide of interest (e.g., SNPs or point mutations), a dinucleotide sequence, or a short sequence of about 5 nucleotides in length, or longer sequences. In some aspects, the methods and compositions comprise a probe comprising a hybridization region comprising an interrogatory region, wherein the hybridization region on the probe is capable of hybridizing to a hybridization region on the target nucleic acid, wherein the hybridization region on the target nucleic acid comprises the region of interest.

In some aspects, detection specificity is increased by blocking the hybridization regions in the target nucleic acid and the probe from hybridizing to each other by providing a blocking strand that prevents the hybridization region on the probe from hybridizing to the hybridization region on the target nucleic acid unless the interrogatory region is complementary to the region of interest. In some embodiments, the blocking strand is hybridized to the hybridization region in the probe. In some embodiments, the blocking strand is hybridized to the hybridization region in the target nucleic acid. In some cases, use of the blocking strand provides certain advantages such as increased specificity and reduced background or false positives for detecting the target sequence (e.g., target SNP).

In some embodiments, the interrogatory region is not complementary to the region of interest, such that the blocking strand is not displaced and the hybridization regions in the target nucleic acid and the probe are unavailable for binding to each other. In other embodiments, the interrogatory region is complementary to the region of interest, such that the blocking strand is displaced and the hybridization regions in the target nucleic acid and the probe are available for binding to each other. In embodiments in which the probe has been displaced, the probe hybridizes to the target nucleic acid, the probe is ligated to itself or to another probe hybridized to the target nucleic acid, and the ligated probe or an amplification product thereof is detected, thereby detecting the region of interest in the target nucleic acid.

In some aspects, the blocking strand and the probe are in different molecules. For instance, the blocking strand may comprise a polynucleotide that is shorter than the probe, e.g., about 15 nucleotides in length. In some aspects, the blocking strand and the probe are in the same molecule. For instance, the blocking strand may be in one of the strands of the stem region in a probe that comprises a metastable stem-loop structure.

Provided herein are methods involving the use of one or more probes (e.g., a circularizable probe such as a padlock probe, or a first probe and a second probe) for analyzing one or more target nucleic acid(s), such as a target nucleic acid (for example, a messenger RNA) present in a cell or a biological sample, such as a tissue sample. Also provided are probes, sets of probes, compositions, kits, systems and devices for use in accordance with the provided methods. In some aspects, the provided methods and systems can be applied to detect, image, quantitate, or determine the presence or absence of one or more target nucleic acid(s) or portions thereof (e.g., presence or absence of sequence variants such as point mutations and SNPs). In some aspects, the provided methods can be applied to detect, image, quantitate, or determine the sequence of one or more target nucleic acid(s), comprising sequence variants such as point mutations and SNPs.

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Methods, compositions, kits, devices, and systems for these in situ assays, comprising spatial genomics and transcriptomics assays, are provided. In some embodiments, a provided method is quantitative and preserves the spatial information within a tissue sample without physically isolating cells or using homogenates.

In some embodiments, through the use of various probe designs (e.g., various circularizable probe, primer, splint, anchor, and/or first and second probe designs), the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the methods disclosed herein involve the use of a circularizable probe or a circularizable probe set comprising probe molecules that are configured to be connected to one another to form a circularized probe. In some aspects, the methods disclosed herein involve the use of one or more probes or probe sets that hybridize to a target nucleic acid, such as an RNA molecule, wherein at least one probe comprises an interrogatory region and is part of a hybridization complex that additionally comprises at least one target nucleic acid and at least one blocking strand. Exemplary probes and probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ hybridization) probe set, and RNA-templated ligation probes. The specific probe or probe set design can vary. In some embodiments, a primary probe (e.g., a DNA probe that directly binds to an RNA target) is amplified through rolling circle amplification. In some embodiments, the primary probes, such as a padlock probe or a probe set that comprises a padlock probe, contain one or more barcodes. In some embodiments, one or more barcodes are indicative of a sequence in the target nucleic acid, such as a single nucleotide of interest (e.g., SNPs or point mutations), a dinucleotide sequence, or a short sequence of about 5 nucleotides in length. In some aspects, the methods disclosed herein is ligation dependent and involve the use of a probe(s) that hybridize to a target nucleic acid and can be ligated, for example, either the ends of the probe can be ligated or two separate probes can be ligated together.

In some aspects, the provided methods involve analyzing (e.g., detecting or determining) one or more sequences present in the polynucleotides and/or in an amplification product, for example in an amplification product of a circularized probe, which comprises one or more barcode sequences. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region (e.g., a single nucleotide) of interest in a target nucleic acid. In some embodiments, the analysis can be used to correlate a sequence detected in an amplification product to a circularizable probe or a first and/or second probe (e.g., via a barcode). In some embodiments, the detection of a sequence in an amplification product can provide information regarding the location and/or identity of an associated target nucleic acid (e.g., hybridized by the circularizable probe or first and second probe) in a sample. In some embodiments, due to amplification of one or more polynucleotides (e.g., a circularizable probe), particular sequences present in the amplification product or complementary sequences thereof can be detected even when a polynucleotide is present at low levels before the amplification. For example, the number of copies of the barcode sequence(s) and/or a complementary sequence thereof is increased by virtue of the amplification of a probe comprising the barcode sequence(s) and/or complementary sequence thereof, thereby enabling specific and sensitive detection of a signal indicative of the identity of a short region (e.g., a single nucleotide) of interest in a target nucleic acid. In particular embodiments, the amplification product is an in situ rolling circle amplification (RCA) product of a circularized probe generated at a location in the sample. In some instances, a signal associated with the amplification product is detected at the location in the biological sample.

In some aspects, the probes (e.g., the circularizable probe or first probe) contain hybridization regions that hybridize to target sites in the target nucleic acids (e.g., DNA or mRNA in a cell). In some aspects, one or more polynucleotides of the set of polynucleotides are used in an extension reaction. In some aspects, one or more polynucleotides of the set of polynucleotides are amplified. In some aspects, the provided methods can be applied for various applications, such as for in situ analysis, comprising in situ detection (e.g., based on hybridization such as sequential hybridization) and/or sequencing of target nucleic acids and multiplexed nucleic acid analysis. In some aspects, the provided methods can be for in situ detection and/or identification of a region (e.g., single nucleotide) of interest in target nucleic acids. In some aspects, the provided methods can be used in a spatial array.

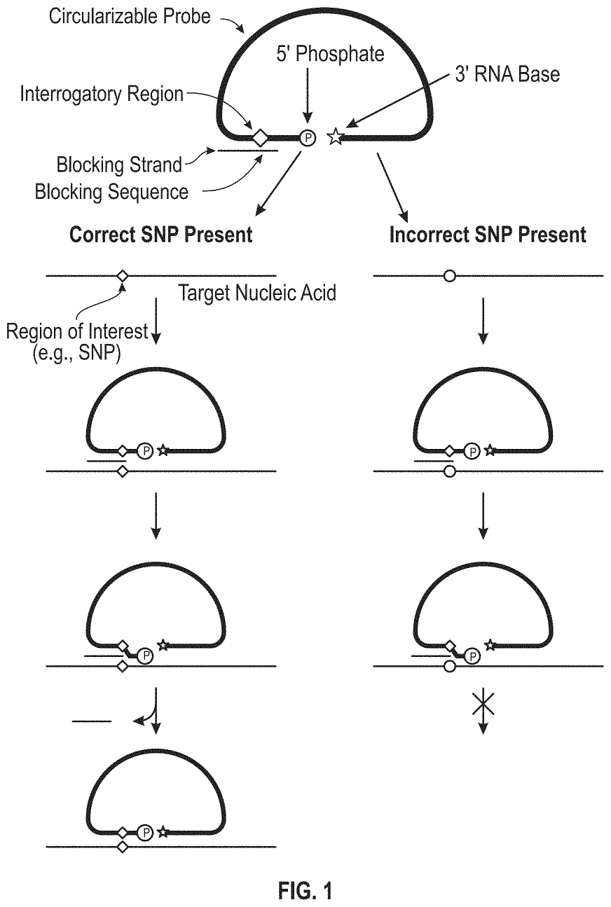

In some embodiments, e.g., as shown in FIGS. 1-4, provided herein are methods for analyzing a biological sample comprising a plurality of target molecules (e.g., RNA) comprising a single nucleotide of interest, the methods comprising: a) contacting the biological sample with a circularizable probe hybridized to a blocking strand, wherein: the blocking strand is hybridized to a hybridization region in the circularizable probe, thereby blocking the hybridization region from hybridizing to a hybridization region in a target molecule, the hybridization region in the circularizable probe comprises an interrogatory nucleotide hybridized to a blocking nucleotide in the blocking strand, and the circularizable probe comprises a toehold region directly linked to the interrogatory nucleotide via a phosphodiester bond; b) allowing hybridization of the toehold region to target molecules in the biological sample, wherein: for a first target molecule in which the single nucleotide of interest is complementary to the interrogatory nucleotide, the blocking strand is displaced and the hybridization region of the circularizable probe is available for hybridizing to the first target molecule, and for a second target molecule in which the single nucleotide of interest is not complementary to the interrogatory nucleotide, the blocking strand is not displaced and the hybridization region of the circularizable probe remains unavailable for hybridizing to the second target molecule; c) allowing molecule(s) of the circularizable probe to dissociate from the second target molecule, under conditions in which molecule(s) of the circularizable probe remain hybridized to the first target molecule; d) circularizing the circularizable probe hybridized to the first target molecule; and e) detecting a rolling circle amplification product of the circularized probe in the biological sample.

In some embodiments, molecule(s) of the circularizable probe are washed from the second target RNA molecule, under conditions in which molecule(s) of the circularizable probe remain hybridized to the first target RNA molecule in biological sample.

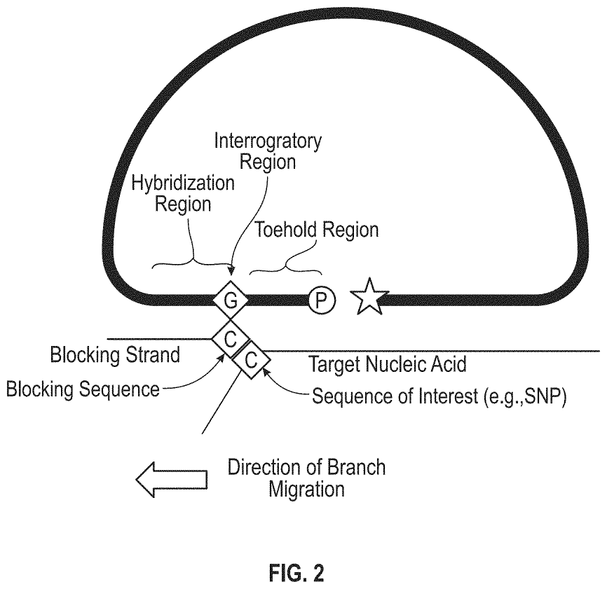

In some embodiments, e.g., as shown in FIGS. 1-2, the blocking strand and the probe are in different molecules. For instance, FIG. 1 shows a complex comprising a circularizable probe (e.g., padlock probe) and a blocking strand is contacted with a biological sample comprising a target nucleic acid (e.g., RNA) comprising a region of interest such as a single nucleotide polymorphism (SNP) of interest. The probe can comprise a 5′ phosphate group and a 3′ RNA base on its 5′ and 3′ ends, respectively, as well as a hybridization region and a toehold region capable of hybridizing to the target nucleic acid. The hybridization region comprises an interrogatory region (e.g., an interrogatory nucleotide) that is complementary to the region of interest. The toehold region can be located between the interrogatory region and the 5′ phosphate group on the probe, and can be directly linked to the interrogatory region via a phosphodiester bond. At the top of FIG. 1, the blocking strand is shown hybridized to the hybridization region of the probe. When the probe and blocking strand contact a target nucleic acid, the toehold region may initially hybridize to the target nucleic acid, but the blocking strand blocks hybridization of the hybridization region in the probe to the target nucleic acid. If the target nucleic acid comprises the SNP of interest, the blocking strand is displaced and the hybridization region of the probe is available to hybridize to the target nucleic acid and the circularizable probe can be ligated using the target nucleic acid as a template (left side of FIG. 1). If the target nucleic acid instead comprises a mismatch at the SNP site with the interrogatory region, the target nucleic acid is not complementary to the probe at that site and the blocking strand is not displaced. The hybridization region of the probe remains blocked by the blocking strand and does not hybridize to the target nucleic acid (right side of FIG. 1).

FIG. 2 shows the circularizable probe (e.g., padlock probe) comprises a hybridization region that is hybridized to a blocking strand, where an interrogatory region (e.g., G) of the circularizable probe is hybridized to a blocking sequence (e.g., C) of the blocking strand. The circularizable probe further comprises a toehold region capable of hybridizing to the target nucleic acid. The exemplary target nucleic acid in this case comprises a sequence of interest (e.g., C) that is complementary to the interrogatory region of the probe. The interrogatory region is adjacent, but not necessarily directly linked by a phosphodiester bond, to the toehold region. As shown in the figure, the target nucleic acid is partially hybridized to the probe on the toehold region, but the blocking strand has not yet been displaced from the hybridization region or the interrogatory region. Branch migration may proceed in the direction shown as the blocking strand is displaced, where the sequence of interest on the target nucleic acid hybridizes to the interrogatory region on the probe.

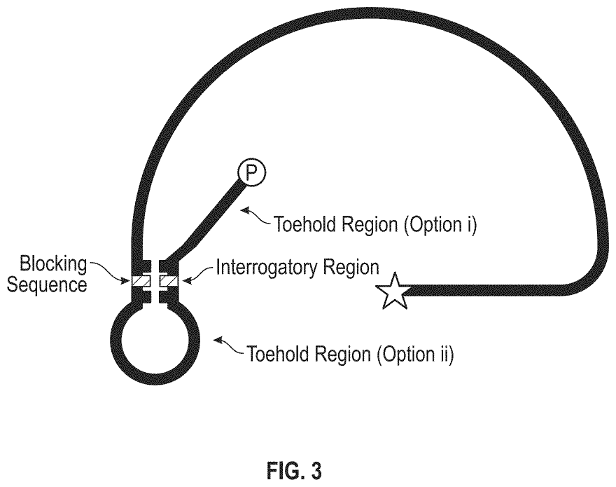

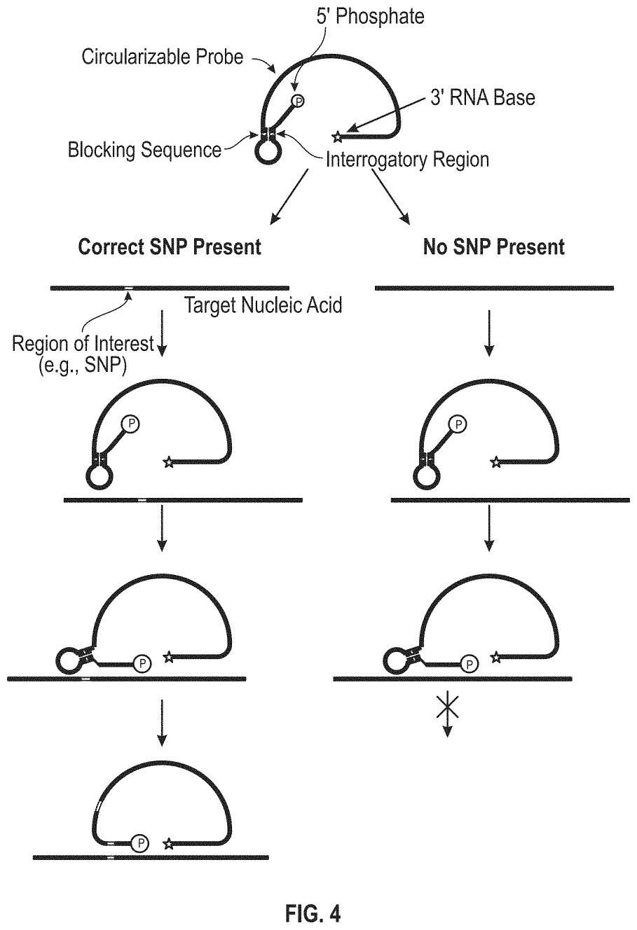

In some embodiments, e.g., as shown in FIGS. 3-4, the blocking strand and the probe are in the same molecule. In FIG. 3, the exemplary blocking sequence and hybridization region in this case form the duplex stem region of the stem-loop structure. Exemplary locations of the toehold region are shown, including outside of the stem-loop structure either towards a terminus of the probe (Toehold region option i) and within the stem-loop structure on a portion of the loop sequence (Toehold region option ii).

In some embodiments, e.g., as shown in FIG. 4, provided herein are methods for analyzing a biological sample comprising a plurality of target molecules (e.g., RNA) comprising a single nucleotide of interest, the methods comprising: a) contacting the biological sample with a circularizable probe comprising a metastable stem-loop structure, wherein: a strand of the stem of the metastable stem-loop structure comprises an interrogatory nucleotide, and the circularizable probe further comprises a toehold region adjacent to the interrogatory nucleotide; b) hybridizing the toehold region to target molecules in the biological sample, wherein: for a first target molecule in which the single nucleotide of interest is complementary to the interrogatory nucleotide, the other strand of the stem is displaced and the strand comprising the interrogatory nucleotide is available for hybridizing to the first target molecule, and for a second target molecule in which the single nucleotide of interest is not complementary to the interrogatory nucleotide, the other strand of the stem is not displaced and the strand comprising the interrogatory nucleotide is unavailable for hybridizing to the second target molecule; c) allowing molecule(s) of the circularizable probe to dissociate from the second target molecule, under conditions in which molecule(s) of the circularizable probe remain hybridized to the first target molecule; d) circularizing the circularizable probe hybridized to the first target molecule; and e) detecting a rolling circle amplification product of the circularized probe in the biological sample. For instance, in FIG. 4, the circularizable probe is contacted with a biological sample comprising a target nucleic acid (e.g., RNA) comprising a region of interest such as a single nucleotide polymorphism (SNP) of interest. The circularizable probe can comprise a 5′ phosphate group and a 3′ RNA base on its 5′ and 3′ ends, respectively, as well as a toehold region (exemplary locations shown in FIG. 3) capable of hybridizing to the target nucleic acid. When the probe contacts a target nucleic acid, the toehold region hybridizes to the target nucleic acid. If the target nucleic acid comprises the SNP of interest such that it is complementary to the interrogatory nucleotide, the stem-loop structure unfolds, displacing the blocking strand from the hybridization region as the hybridization region hybridizes to the target nucleic acid (left side of FIG. 4). However, if the target nucleic acid does not comprise a SNP complementary to the interrogatory nucleotide, the stem-loop structure remains stable and the blocking strand is not displaced. The hybridization region of the probe remains blocked by the blocking strand and does not hybridize to the target nucleic acid (right side of FIG. 4).

In some embodiments, molecule(s) of the circularizable probe that do not hybridize to a target via the loop and/or stem sequences or portion(s) thereof are washed from the biological sample, under conditions in which molecule(s) of the circularizable probe that hybridize via the loop and/or stem sequences or portion thereof remain hybridized to the target nucleic acid in biological sample.

In any of the preceding embodiments, the circularizable probe may be circularized using a template such as, for example, the target nucleic acid or a splint hybridized to the target nucleic acid.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA. In some embodiments, the target nucleic acid comprises RNA. In some embodiments, the probe comprises DNA. In some embodiments, the target nucleic acid is RNA and the probe is DNA.

In some aspects, the provided methods are employed for in situ analysis of target nucleic acids, for example for in situ sequencing or multiplexed analysis in intact tissues or a sample with preserved cellular or tissue structure. In some aspects, the provided methods can be used to detect or determine the identity or amount in situ of single nucleotides of interest in target nucleic acids, for instance of single nucleotide polymorphisms of genes of interest.

In some aspects, the provided methods involve a step of contacting, or hybridizing, one or more polynucleotides, such as any of the probes described herein, to a cell or a sample containing a target nucleic acid with a region (e.g., single nucleotide) of interest in order to form a hybridization complex. In some aspects, the provided methods comprise one or more steps of ligating the polynucleotides, for instance of ligating the ends of a circularizable probe to form a circularized probe and/or ligating a first probe and second probe to form a ligated first-second probe product. In some aspects, the provided methods involve a step of amplifying one of the polynucleotides (e.g., a circularizable probe or a circularized probe produced therefrom), to generate an amplification product. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve detecting the ligation product of a first and second probe with or without amplification. In some aspects, the first and/or second probes comprise an overhang sequence that provides a landing pad for hybridization of one or more additional probes for detection of the ligated first-second probe. In some aspects, the provided methods involve performing one or more of the steps described herein, simultaneously and/or sequentially.

Particulars of the steps of the methods can be carried out as described herein, for example in Sections II-VI; and/or using any suitable processes and methods for carrying out the particular steps.

### II. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

(1) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

(ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

(iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

(v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, propidium iodide, rhodamine, safranine, or derivatives thereof. In some embodiments, the sample may be stained with haematoxylin and eosin (H & E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable method of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

(vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-crosslinking

In some embodiments, the biological sample is reversibly cross-linked prior to or during an in situ assay round. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method known in the art. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of species (such as probes) in the sample and/or the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, TritonX100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods are known in the art. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

(ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, one or more nucleic acid probes can be used to hybridize to a target nucleic acid (e.g., cDNA or RNA molecule, such as an mRNA) and ligated in a templated ligation reaction (e.g., RNA-templated ligation (RTL) or DNA-templated ligation (e.g., on cDNA)) to generate a product for analysis. In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(1) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, the methods provided herein for analyzing endogenous analytes with a region of interest in a sample can be combined with methods for detecting other analytes using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a circularizable probe such as a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation (RTL) probes. The specific probe or probe set design can vary.

(b) Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agents. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° NTM DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect ligation” means that the ends of the polynucleotides are separated by one or more intervening nucleotides or “gaps”. In some embodiments, said ends are not ligated directly to each other, but ligation instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product is a primer extension product of an analyte, a labelling agent, a probe or probe set bound to the analyte (e.g., a circularizable probe bound to genomic DNA, mRNA, or cDNA), or a probe or probe set bound to the labelling agent (e.g., a circularizable probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer may, in some cases, refer to a primer binding sequence. A primer extension reaction generally refers to any method where two nucleic acid sequences become linked (e.g., hybridized) by an overlap of their respective terminal complementary nucleic acid sequences (for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (amplicon) containing multiple copies of the cDNA. Techniques for rolling circle amplification (RCA) are known in the art, such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Examples of the modified nucleotides comprise amine-modified nucleotides, for example, for anchoring or cross-linking of the generated amplification product (e.g., nanoball) to a scaffold, to cellular structures and/or to other amplification products (e.g., other nanoballs). In some aspects, the amplification products comprise a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, U.S. Pat. Nos. 10,138,509, 10,266,888, 10,494,662, 10,545,075, US 2016/0024555, US 2018/0251833 and US 2017/0219465, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix, thereby preserving their spatial relationship and any information inherent thereto. For example, if the amplification products are those generated from DNA or RNA within a cell embedded in the matrix, the amplification products can also be functionalized to form covalent attachment(s) to the matrix, preserving their spatial information within the cell and thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe or probe set disclosed herein may be a hybridization complex formed of a cellular nucleic acid in a sample and an exogenously added nucleic acid probe. The exogenously added nucleic acid probe(s) may comprise an overhang that does not hybridize to the cellular nucleic acid but hybridizes to another probe (e.g., detectably labelled probes). The exogenously added nucleic acid probe may be optionally ligated to a cellular nucleic acid molecule or another exogenous nucleic acid molecule. In other examples, a product comprising a target sequence for a probe disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein may a probe hybridizing to an RCP. The probe may comprise an overhang that does not hybridize to the RCP but hybridizes to another probe. The probe may be optionally ligated to a cellular nucleic acid molecule or another probe, e.g., an anchor probe that hybridize to the RCP.

C. Target Sequences

A target sequence for a probe disclosed herein may be comprised in any analyte disclose herein, including an endogenous analyte (e.g., a viral or cellular nucleic acid), a labelling agent, or a product of an endogenous analyte and/or a labelling agent.

In some aspects, the target sequence is in a target nucleic acid. In some embodiments, the target nucleic acid comprises DNA and/or RNA, such as, for example, mRNA. In some aspects, the target nucleic acid comprises a region of interest. A region of interest may include any sequence, and may comprise, for example, one or more single nucleotide polymorphisms (SNPs).

In some aspects, one or more portions of the target sequence are capable of hybridizing to one or more portions of a probe or probes. In some embodiments, the region of interest on a target nucleic acid is complementary to a hybridization region on a probe. The hybridization region on the probe may comprise one or more interrogatory nucleotides. In some embodiments, the region of interest on a target nucleic acid is complementary to the interrogatory nucleotide(s) on the probe. In some embodiments, the region of interest on a target nucleic acid is complementary to all but one or more interrogatory nucleotides on the probe. In some embodiments, the region of interest on a target nucleic acid is not complementary to the interrogatory nucleotide(s) on the probe.

In some aspects, a sequence adjacent to the region of interest on a target nucleic acid comprises a toehold region that is complementary to a toehold region on a probe.

In some aspects, the target sequence is hybridized to a blocking strand. A blocking strand can be part of the same molecule as a probe, or on a different molecule. In some embodiments, the target sequence is hybridized to a blocking strand before being contacted with a probe. In some embodiments, both the probe and target sequence are hybridized to separate blocking strands. In some aspects, only the probe is hybridized to a blocking strand. In some embodiments, the blocking strand(s) is/are displaced, allowing hybridization between the probe and the target sequence. In some embodiments, the blocking strand(s) is/are not displaced, and the probe and the target are unavailable for hybridizing to each other.

In some aspects, the target sequence is hybridized to a probe. In some embodiments, hybridization between the probe and the target is stable. In some embodiments, hybridization between the probe and the target is unstable. Hybridization is considered “stable” when it is thermodynamically favoured, such that a stably hybridized probe and target pair remain hybridized to each other. Hybridization is considered “unstable” when a hybridized probe and target pair dissociate from each other under conditions in which a stable hybridization would remain hybridized. In some embodiments, hybridization between the probe and the target is sufficiently stable for ligation to occur. In some embodiments, probes are ligated only when they are stably hybridized to a target. In some embodiments, probes are not ligated when they are either not hybridized to a target or only unstably hybridized to a target.

In some embodiments, any of the hybridization regions and/or regions of interest contained in the target nucleic acid are between or between about 5 and 40 nucleotides in length. In some embodiments, the target hybridization regions and/or regions of interest are between or between about 5 and 15 nucleotides in length. In some embodiments, the target hybridization regions and/or regions of interest are between or between about 15 and 20 nucleotides in length. In some embodiments, the target hybridization regions and/or regions of interest are between or between about 20 and 25 nucleotides in length. In some embodiments, the target hybridization regions and/or regions of interest are between or between about 25 and 30 nucleotides in length. In some embodiments, the target hybridization regions and/or regions of interest are between or between about 30 and 35 nucleotides in length. In some embodiments, the target hybridization regions and/or regions of interest are between or between about 25 and 30 nucleotides in length. In some embodiments, the target hybridization regions and/or regions of interest are between or between about 35 and 40 nucleotides in length.

In some embodiments, the region of interest of the target nucleic acid comprises one, two, three, four, five, or more nucleotide positions. In some embodiments, the region of interest can comprise a single nucleotide of interest, an alternatively spliced region, a deletion, and/or a frameshift. In some embodiments, the single nucleotide of interest is selected from the group consisting of a single-nucleotide polymorphism (SNP), a single-nucleotide variant (SNV), a single-nucleotide substitution, a point mutation, a single-nucleotide deletion, and a single-nucleotide insertion. In some embodiments, provided herein are methods and compositions for analyzing two or more sequences of a region of interest (e.g., a first and second sequence of the region of interest). In some embodiments, the first and second sequences of the region of interest are different at one, two, three, four, five, or more nucleotide positions. In some embodiments, the first and/or second sequences of the region of interest comprise a single nucleotide of interest, an alternatively spliced region, a deletion, and/or a frameshift. In some embodiments, the second sequence of the region of interest is a variant of the first sequence of the region of interest, or vice versa. In some embodiments, the variant comprises a single-nucleotide polymorphism (SNP), a single-nucleotide variant (SNV), a single-nucleotide substitution, a point mutation, a single-nucleotide deletion, or a single-nucleotide insertion.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub. 20210164039, which are hereby incorporated by reference in their entirety.

### III. Polynucleotides and Hybridization Complexes

Disclosed herein in some aspects are nucleic acid probes and/or probe sets that are introduced into a cell or used to otherwise contact a biological sample such as a tissue sample. In some aspects, the nucleic acid probes and/or probe sets comprise a hybridization region comprising an interrogatory region, wherein the hybridization region on the probe is capable of hybridizing to a hybridization region on the target nucleic acid, wherein the hybridization region on the target nucleic acid comprises a region of interest. In some aspects, the probes and/or probe sets are designed such that detection specificity and stringency is increased by blocking the hybridization regions in the target nucleic acid and the probe from hybridizing to each other by providing a blocking strand that prevents the hybridization region on the probe from hybridizing to the hybridization region on the target nucleic acid unless the interrogatory region is complementary to the region of interest. In some embodiments, the blocking strand is hybridized to the hybridization region in the probe. In some embodiments, the blocking strand is hybridized to the hybridization region in the target nucleic acid.

The probes may comprise any of a variety of entities that can hybridize to a nucleic acid, typically by Watson-Crick base pairing, such as DNA, RNA, LNA, PNA, etc., depending on the application. The nucleic acid probe(s) typically contains a hybridization region that is able to bind to at least a portion of a target nucleic acid, in some embodiments specifically. The nucleic acid probe may be able to bind to a specific target nucleic acid (e.g., an mRNA, or other nucleic acids as discussed herein). In some embodiments, the nucleic acid probes may be detected using a detectable label, and/or by using secondary nucleic acid probes able to bind to the nucleic acid probes. In some embodiments, the nucleic acid probes are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease for cleavage).

Provided herein are methods involving the use of one or more probes (e.g., a circularizable probe) for analyzing one or more target nucleic acid(s), such as a target nucleic acid (for example, a messenger RNA) present in a cell or a biological sample, such as a tissue sample. Also provided are probes, sets of probes, compositions, kits, systems and devices for use in accordance with the provided methods. In some aspects, the provided methods and systems can be applied to detect, image, quantitate, or determine the presence or absence of one or more target nucleic acid(s) or portions thereof (e.g., presence or absence of sequence variants such as point mutations and SNPs). In some aspects, the provided methods can be applied to detect, image, quantitate, or determine the sequence of one or more target nucleic acid(s), comprising sequence variants such as point mutations and SNPs.

In some aspects, a target nucleic acid disclosed herein comprises any polynucleotide nucleic acid molecule (e.g., DNA molecule; RNA molecule, modified nucleic acid, etc.) for assessment in accordance with the provided embodiments, such as a polynucleotide present in a cell. In some embodiments, the target nucleic acid is a coding RNA (e.g., mRNA). The target may, in some embodiments, be a single RNA molecule. In other embodiments, the target may be at least one RNA molecule, e.g., a group of 2, 3, 4, 5, 6 or more RNA molecules. These RNA molecules may differ in molecule type, and/or may differ in sequence. In some embodiments, the target nucleic acid is, for example, a non-coding RNA (e.g., tRNA, rRNA, microRNA (miRNA), mature miRNA or immature miRNA). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, e.g., have a differing abundance, within a cell population, wherein the methods of the invention allow profiling and comparison of the expression levels of nucleic acids, comprising but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like.

In some aspects, the methods provided herein are used to analyze a target nucleic acid, e.g., a messenger RNA molecule. In some embodiments, the target nucleic acid is an endogenous nucleic acid present in a biological sample. In some embodiments, the target nucleic acid is present in a cell in a tissue, for instance in a tissue sample or tissue section. In some embodiments, the tissue sample is an intact tissue sample or a non-homogenized tissue sample. In some embodiments, the tissue sample is a fresh tissue sample. In some embodiments, the tissue has previously been processed, e.g., fixed, embedded, frozen, or permeabilized using any of the steps and/or protocols described in Section II. In some embodiments, the target nucleic acid is an exogenous nucleic acid contacted with a biological sample.

In some aspects, the provided embodiments can be employed for in situ detection and/or sequencing of a target nucleic acid in a cell, e.g., in cells of a biological sample or a sample derived from a biological sample, such as a tissue section on a solid support, such as on a transparent slide.

In some aspects, provided herein are in situ assays using microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a method for spatially profiling analytes such as the transcriptome or a subset thereof in a biological sample is provided. Methods, compositions, kits, devices, and systems for these in situ assays, comprising spatial genomics and transcriptomics assays, are provided. In some embodiments, a provided method is quantitative and preserves the spatial information within a tissue sample without physically isolating cells or using homogenates. In some embodiments, the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the methods disclosed herein involve the use of one or more probes or probe sets that hybridize to a target nucleic acid, such as an RNA molecule. A circularizable probe or probe set can be any nucleic acid molecule or set of nucleic acid molecules that hybridize to one or more other nucleic acids such that the ends of the nucleic acid molecule or nucleic acid molecules are juxtaposed or are in proximity for ligation to form a circularized probe (e.g., by ligation with or without gap filling). For example, a circularizable probe may be a padlock probe with ends that can be ligated to form a circularized padlock probe. Specific probe designs can vary depending on the application. For instance, probes or probe sets described herein can comprise a circularizable probe that does not require gap filling to circularize upon hybridization to a template (e.g., a target nucleic acid and/or a probe such as a splint), a gapped circularizable probe (e.g., one that requires gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504 incorporated herein by reference in their entireties, and probes for Proximity Ligation Assay (PLA) for the simultaneous detection and quantification of nucleic acid molecules and protein-protein interactions), or any suitable combination thereof. In some embodiments, a probe disclosed herein can comprise a probe that is ligated to itself or another probe using DNA-templated and/or RNA-templated ligation. In some embodiments, a probe disclosed herein can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template, and the probes can comprise RNA-templated ligation probes. In some embodiments, a probe disclosed herein can comprise a padlock-like probe or probe set, such as one described in US 2019/0055594, US 2021/0164039, US 2016/0108458, or US 2020/0224243, each of which is incorporated herein by reference in its entirety. Any suitable combination of the probe designs described herein can be used.

In some embodiments, probes or probe sets described herein can comprise two or more parts. In some cases, a probe can comprise one or more features of and/or be modified based on: a split FISH probe or probe set described in WO 2021/167526A1 or Goh et al., “Highly specific multiplexed RNA imaging in tissues with split-FISH,” Nat Methods 17(7):689-693 (2020), which are incorporated herein by reference in their entireties; a Z-probe or probe set, such as one described in U.S. Pat. No. 7,709,198 B2, U.S. Pat. No. 8,604,182 B2, U.S. Pat. No. 8,951,726 B2, U.S. Pat. No. 8,658,361 B2, or Tripathi et al., “Z Probe, An Efficient Tool for Characterizing Long Non-Coding RNA in FFPE Tissues,” Noncoding RNA 4(3):20 (2018), which are incorporated herein by reference in their entireties; an HCR initiator or amplifier, such as one described in U.S. Pat. No. 7,632,641 B2, US 2017/0009278 A1, U.S. Pat. No. 10,450,599 B2, Dirks and Pierce, “Triggered amplification by hybridization chain reaction,” PNAS 101(43):15275-15278 (2004), Chemeris et al., “Real-time hybridization chain reaction,” Dokl. Biochem 419:53-55 (2008), Niu et al., “Fluorescence detection for DNA using hybridization chain reaction with enzyme-amplification,” Chem Commun (Camb) 46(18):3089-91 (2010), Choi et al., “Programmable in situ amplification for multiplexed imaging of mRNA expression,” Nat Biotechnol 28(11):1208-12 (2010), Song et al., “Hybridization chain reaction-based aptameric system for the highly selective and sensitive detection of protein,” Analyst 137(6):1396-401 (2012), Choi et al., “Third-generation in situ hybridization chain reaction: multiplexed, quantitative, sensitive, versatile, robust,” Development 145(12): dev165753 (2018), or Tsuneoka and Funato, “Modified in situ Hybridization Chain Reaction Using Short Hairpin DNAs,” Front Mol Neurosci 13:75 (2020), which are incorporated herein by reference in their entireties; a PLAYR probe or probe set, such as one described in US 2016/0108458 A1 or Frei et al., “Highly multiplexed simultaneous detection of RNAs and proteins in single cells,” Nat Methods 13(3):269-75 (2016), which are incorporated herein by reference in their entireties; a PLISH probe or probe set, such as one described in US 2020/0224243 A1 or Nagendran et al., “Automated cell-type classification in intact tissues by single-cell molecular profiling,” eLife 7:e30510 (2018), which are incorporated herein by reference in their entireties; a RollFISH probe or probe set such as one described in Wu et al., “RollFISH achieves robust quantification of single-molecule RNA biomarkers in paraffin-embedded tumor tissue samples,” Commun Biol 1, 209 (2018), which is hereby incorporated by reference in its entirety; a MERFISH probe or probe set, such as one described in US 2022/0064697 A1 or Chen et al., “Spatially resolved, highly multiplexed RNA profiling in single cells,” Science 348(6233):aaa6090 (2015); or a primer exchange reaction (PER) probe or probe set, such as one described in US 2019/0106733 A1, each of which is hereby incorporated by reference in its entirety.

In some embodiments, probes or probe sets described herein comprise one or more features and/or is modified to allow for generation and detection of a signal that does not comprise a polymerase-catalyzed nucleic acid amplification step (e.g., the signal can be an smFISH signal). In some instances, the probes or probe sets described herein for each target comprises probes directly hybridize to multiple regions (e.g., sequences) of the same transcript. In some embodiments, the probes or probe sets described herein comprise a circular probe or circularizable probe or probe set comprises one or more features and/or is modified to allow for generation and detection of a second signal that comprises an amplification step (e.g., extension and/or amplification catalyzed by a polymerase).

In some embodiments, at least one probe is a circularizable probe. In some embodiments, the circularizable probe comprises asymmetric arms that hybridize to a nucleic acid molecule. Exemplary probes may be based on a circularizable probe, a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some embodiments, a primary probe (e.g., a DNA probe that directly binds to an RNA target) is amplified through rolling circle amplification. In some embodiments, the primary probes, such as a padlock probe or a probe set that comprises a padlock probe, contain one or more barcodes. In some embodiments, one or more barcodes are indicative of a sequence in the target nucleic acid, such as a single nucleotide of interest (e.g., SNPs or point mutations), a dinucleotide sequence, or a short sequence of about 5 nucleotides in length.

In some aspects, the provided methods involve analyzing, e.g., detecting or determining, one or more sequences present in the polynucleotides and/or in an amplification product, such as in an amplification product of a circularized probe (e.g., padlock probe), which comprises one or more barcode sequences. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region (e.g., a single nucleotide) of interest in a target nucleic acid. In some embodiments, the analysis can be used to correlate a sequence detected in an amplification product to a circularizable probe (e.g., via a barcode). In some embodiments, the detection of a sequence in an amplification product can provide information regarding the location and/or identity of an associated target nucleic acid (e.g., hybridized by the circularizable probe) in a sample. In some embodiments, due to amplification of one or more polynucleotides (e.g., a circularizable probe), particular sequences present in the amplification product or complementary sequences thereof can be detected even when a polynucleotide is present at low levels before the amplification. For example, the number of copies of the barcode sequence(s) and/or a complementary sequence thereof is increased by virtue of the amplification of a probe comprising the barcode sequence(s) and/or complementary sequence thereof, thereby enabling specific and sensitive detection of a signal indicative of the identity of a short region (e.g., a single nucleotide) of interest in a target nucleic acid. In particular embodiments, the amplification product is an in situ rolling circle amplification (RCA) product of a circularized probe.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA. In some embodiments, the target nucleic acid comprises RNA. In some embodiments, the probe comprises DNA. In some embodiments, the target nucleic acid is RNA and the probe comprises DNA.

In some aspects, the provided methods are employed for in situ analysis of target nucleic acids, for example for in situ sequencing or multiplexed analysis in intact tissues or a sample with preserved cellular or tissue structure. In some aspects, the provided methods can be used to detect or determine the identity or amount in situ of single nucleotides of interest in target nucleic acids, for instance of single nucleotide polymorphisms of genes of interest. In some aspects, the provided methods are employed for analysis of target nucleic acids in a spatial array, as discussed, for example in Section V.

In some aspects, the provided methods involve a step of contacting, or hybridizing, one or more polynucleotides, such as any of the probes described herein, to a cell or a sample containing a target nucleic acid with a region (e.g., single nucleotide) of interest in order to form a hybridization complex. In some aspects, the provided methods comprise one or more steps of ligating the polynucleotides, for instance of ligating the ends of a circularizable probe to form a circularized probe. In some aspects, the provided methods involve a step of amplifying one of the polynucleotides (e.g., a circularizable probe or a circularized probe produced therefrom), to generate an amplification product. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve performing one or more of the steps described herein, simultaneously and/or sequentially.

In some embodiments, a circularizable probe disclosed herein comprise 1, 2, 3, 4, 5, 6, 7, 8, 9, 10, 11, 12, 13, 14, 15 or more, 20 or more, 32 or more, 40 or more, or 50 or more barcode sequences. The barcode sequences may be positioned anywhere within the nucleic acid probe. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of nucleic acid probes is less than the number of distinct targets (e.g., nucleic acid analytes and/or protein analytes) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes. In some embodiments, the barcode sequences or any subset thereof in the population of nucleic acid probes can be independently and/or combinatorially detected and/or decoded.

As an illustrative example, a first probe may contain a first hybridization region, a first barcode sequence, and a second barcode sequence, while a second, different probe may contain a second hybridization region (that is different from the first hybridization region in the first probe), the same first barcode sequence as in the first probe, but a third barcode sequence instead of the second barcode sequence. Such probes may thereby be distinguished by determining the various barcode sequence combinations present or associated with a given probe at a given location in a sample.

In some aspects, the methods provided herein comprise use of one or more polynucleotides, for example at least one circularizable probe such as a padlock probe, a SNAIL or SNAIL-like probe, and/or circularizable or linear probes generated from RNA-templated ligation (RTL) of probe molecules, wherein the probe or probes comprise a hybridization region comprising an interrogatory region. In some aspects, the polynucleotide further comprises an interrogatory nucleotide. In some aspects, the polynucleotide further comprises a toehold region adjacent to the interrogatory region. In some aspects, the toehold region is directly linked to the interrogatory nucleotide via a phosphodiester bond. In some aspects, the polynucleotides further comprise a metastable stem-loop structure. In some aspects, the hybridization region is hybridized to a blocking strand. In some aspects, the blocking strand and the probe are in different molecules. In some aspects, the blocking strand and the probe are in the same molecule.

The metastable stem-loop structure in the context of a probe for detecting a region of interest in a target nucleic acid is a structure is stable in the absence of a complementary target nucleic acid, such that the presence of a complementary target nucleic acid favors the destabilization of the stem-loop structure and the hybridization of a sequence in the stem region with the target nucleic acid, and may be used interchangeably with terms such as “metastable hairpin” and the like. In some embodiments, the stem-loop structure is metastable such that subsequent steps such as destabilization of the stem-loop structure, circularizable probe hybridization and ligation, and/or amplification of the ligated circularizable probe are dependent upon the recognition of the target nucleic acid of interest (e.g., a SNP in the target nucleic acid) by a sequence in the stem-loop structure.

The term “blocking strand” encompasses but is not limited to concepts from the field of strand displacement (such as an “incumbent strand” as described, for example, in Wang et al., PNAS (2018), 52, E12182-E12191, and/or a “protector strand,” both of which are commonly used in the field of strand displacement). For example, a “blocking strand” may hybridize to (or “block”) a region on a different molecule or on the same molecule as the blocking strand. As such, a strand portion of the stem region of a stem-loop structure, for example, may serve as a “blocking strand” for a portion of the other strand of the stem.

In some aspects, the probe or probes comprise one or more interrogatory regions. In some embodiments, any of the interrogatory regions contained in the probes are between or between about 1 and 50 nucleotides in length. In some embodiments, any of the interrogatory regions contained in the probes are between or between about 1 and 40 nucleotides in length. In some embodiments, any of the interrogatory regions contained in the probes are between or between about 1 and 30 nucleotides in length. In some embodiments, any of the interrogatory regions contained in the probes are between or between about 1 and 20 nucleotides in length. In some embodiments, any of the interrogatory regions contained in the probes are between or between about 1 and 10 nucleotides in length. In some embodiments, any of the interrogatory regions contained in the probes are between or between about 1 and 5 nucleotides in length. In some embodiments, the interrogatory region can comprise 1 nucleotide for detecting a single nucleotide of interest.

In some embodiments, there is provided a first probe (e.g., a first circularizable probe or a first probe A) for detecting a first nucleotide of interest (e.g., a SNP) and a second probe (e.g., a second circularizable probe or a first probe B) for detecting an alternate nucleotide of interest (e.g., an alternate SNP). For example, a region of interest may comprise one of two variants, the first nucleotide of interest is C, and the alternate nucleotide of interest is A. However, the methods and compositions provided herein can discriminate between any two nucleotides. For example, the first probe can comprise a first barcode sequence, a competing region, and a first interrogatory nucleotide (e.g., G) capable of basepairing with a first nucleotide of interest (e.g., C), and the second probe can comprise a second barcode sequence, a competing region, and an alternate interrogatory nucleotide (e.g., T) capable of basepairing with an alternate nucleotide of interest (e.g., A). In some embodiments, if the interrogatory nucleotide of a probe matches with the nucleotide of interest, the interrogatory nucleotide will hybridize to the nucleotide of interest with higher affinity or at a higher frequency than the competing region for the nucleotide of interest, thereby out-competing the competing region for hybridization to the nucleotide of interest. Binding of the interrogatory region to the region of interest (e.g., a SNP) allows ligation of probe(s) (e.g., ligation of the ends of the circularizable probe to form a circularized probe, or ligation of the first probe and second probe to form a ligated first-second probe). In some embodiments, the concentration of each of the provided probes contacted with the sample can be tuned for sensitivity and selectivity.

In some embodiments, disclosed herein is a method for analyzing a biological sample, comprising contacting the biological sample with a circularizable probe, wherein the circularizable probe comprises a 5′ hybridization region and a 3′ hybridization region for hybridizing to adjacent first and second regions, respectively, in a target nucleic acid, and the 5′ hybridization region and the 3′ hybridization region are different in lengths. In some embodiments, the 5′ hybridization region and/or the 3′ hybridization region comprises an interrogatory sequence for interrogating a region of interest in the first or second region, respectively. In some embodiments, the shorter one of the 5′ hybridization region and the 3′ hybridization region comprises the interrogatory sequence, such as an interrogatory nucleotide for a single nucleotide of interest in the target nucleic acid. In some embodiments, the sequence of interest in a first molecule of the target nucleic acid is complementary to the interrogatory sequence, whereas the sequence of interest in a second molecule of the target nucleic acid comprises a mismatch with the interrogatory sequence. In some embodiments, the mismatch is not at the 5′ or 3′ terminal nucleotide of the circularizable probe. In some embodiments, the method further comprises ligating the 5′ and the 3′ hybridization regions of the circularizable probe hybridized to the first molecule of the target nucleic acid to form a circularized probe, whereas under the same or similar conditions the 5′ and the 3′ hybridization regions of the circularizable probe hybridized to the second molecule of the target nucleic acid are not ligated. In some embodiments, the method further comprises detecting the circularized probe in the biological sample. In some embodiments, a signal associated with the circularized probe is detected in situ at the location of the first molecule of the target nucleic acid. In some embodiments, a circularizable probe comprises a 5′ hybridization region and a 3′ hybridization region each comprising an interrogatory region for detecting a different nucleotide of interest (e.g., a SNP). In some aspects, each hybridization region of the circularizable probe may be hybridized to a blocking strand.

In some embodiments, provided herein is a method for analyzing a biological sample, comprising contacting the biological sample with a circularizable probe or probe set. In some embodiments, the circularizable probe or probe set comprises a linear oligonucleotide sequence that, upon hybridization to a target nucleic acid, such as an RNA molecule, forms a probe that can be circularized. The circularizable probe can be circularized via ligation (or primer extension followed by ligation) using the target RNA and/or the DNA splint as a template. The circularizable probe or probe set can comprise asymmetric 5′ and 3′ hybridization regions that hybridize to adjacent first and second target regions, respectively, in the target RNA. The 5′ hybridization region can be longer than the 3′ hybridization region, or vice versa. The first target region can be longer than the second target region, or vice versa. The internal interrogatory sequence can be on the 5′ or 3′ arm and does not participate in a subsequent ligation step. The circularizable probe or probe set can comprise a blocking strand that hybridizes to the probe or probes and/or to the target nucleic acid, thereby blocking the target nucleic acid and the probe from hybridizing to each other unless the blocking strand is displaced.

In some embodiments, an interrogatory nucleotide can be the first nucleotide at the branch point. In some embodiments, a 5′ or 3′ terminal nucleotide of a blocking sequence hybridizes to the interrogatory nucleotide of a probe, and upon hybridization of the target nucleic acid to the probe, a single nucleotide of interest in the target nucleic acid displaces the 5′ or 3′ terminal nucleotide and hybridizes to the interrogatory nucleotide, thereby migrating the branch point. In some embodiments, mismatch between the interrogatory nucleotide and the single nucleotide of interest at the branch point prevents initiation of strand displacement and branch migration.

In some embodiments, an interrogatory nucleotide can be internal, that is, not at the branch point when branch migration is initiated. In some embodiments, strand displacement may be initiated but branch migration is stalled when there is a mismatch between the interrogatory nucleotide and the single nucleotide of interest. In some embodiments, stalled branch migration may favor re-hybridization of the blocking strand.

In any of the embodiments herein, the presence of at least one base in the interrogatory region that is not complementary to the region of interest may limit displacement of the blocking strand. In some embodiments, displacement of the blocking strand occurs uni-directionally. In some embodiments, displacement of the blocking strand occurs bi-directionally.

In some embodiments, the blocking strand can be in the same molecule as the probe. For instance, the probe may comprise a metastable stem-loop structure, in which one arm of the stem of the stem-loop structure acts as the blocking strand. In some embodiments, the blocking strand can be in a different molecule from the probe. For instance, the blocking strand may be a separate polynucleotide, such as, for example, a 15 bp long oligonucleotide, that hybridizes to the probe or to the target nucleic acid. In any of the embodiments herein in which the blocking strand hybridizes to the probe, the blocking strand may hybridize to any region of the probe, such as on either arm of a circularizable probe.

In any of the embodiments herein, the interrogatory region may comprise a variety of different locations on the probe. For example, the interrogatory region may comprise, e.g., the 2nd, 3rd, 4th, 5th, 6th, 7th, 8th, 9th, and/or 10th positions from the ligation site on the probe arm and/or either or both ends of the region that hybridizes to the blocking strand, though it is not limited to any of those particular locations.

For probes comprising a metastable stem-loop structure, the loop region of the stem-loop structure may be, e.g., roughly 15 bp long and each strand of the stem region may be, e.g., roughly 5-10 bp long, but other lengths may be used as well, so long as the probe is capable of forming a metastable stem-loop structure and the stem is long enough to comprise a hybridization region comprising an interrogatory region that is capable of hybridizing with the region of interest on the target nucleic acid. For instance, the loop region may alternatively be significantly longer than the stem. In some embodiments, the hairpin is about 15 and about 25 bp in length, such as 15, 16, 17, 18, 19, 20, 21, 22, 23, 24, or 25 bp in length. In some embodiments, the loop is about 10 to about 20 bp in length, such as 10, 11, 12, 13, 14,15, 16, 17, 18, 19, or 20 bp in length. In some embodiments, the stem is about 5 to about 10 bp in length, such as 5, 6, 7, 8, 9, or 10 bp in length. In some embodiments, the loop is 15 bp long and the stem is 5 bp long.

The internal interrogatory sequence can be in the shorter one of the 5′ hybridization region and the 3′ hybridization region. In some embodiments, the internal interrogatory sequence can be in the longer one of the 5′ hybridization region and the 3′ hybridization region. In any of the embodiments herein, the 5′ hybridization region of a circularizable probe herein can be shorter or longer than the 3′ hybridization region. In any of the embodiments herein, the 5′ hybridization region and the 3′ hybridization region can be independently 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more nucleotides in length. In some embodiments, the 5′ hybridization region is between about 5 and about 20 nucleotides in length, while the 3′ hybridization region is between about 10 and about 40 nucleotides in length. In some embodiments, the 5′ hybridization region is between about 10 and about 15 nucleotides in length, while the 3′ hybridization region is between about 15 and about 30 nucleotides in length. In some embodiments, the 5′ hybridization region is between about 8 and about 12 nucleotides in length, while the 3′ hybridization region is between about 16 and about 24 nucleotides in length. In some embodiments, the 5′ hybridization region is about 10 nucleotides in length, while the 3′ hybridization region is about 20 nucleotides in length.

In some embodiments, the blocking strand is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more nucleotides in length. In some embodiments, the blocking strand is shorter than hybridization region of the circularizable probe that is complementary to the target nucleic acid. In some embodiments, the blocking strand is at least 1, 2, 3, 4, 5, 6, 7, 8, 9, 10, 11, 12, 13, 14, 15, 16, 17, 18, 19, 20, or more nucleotides shorter than the hybridization region of the circularizable probe. In some embodiments, the blocking strand is between 2 and 5 nucleotides shorter than the hybridization region. In some embodiments, the blocking strand is between 5 and 10 nucleotides shorter than the hybridization region. In some embodiments, the blocking strand is between 10 and 15 nucleotides shorter than the hybridization region.

In any of the embodiments herein, the lengths of the 5′ hybridization region and the 3′ hybridization region can differ by 1, 2, 3, 4, 5, 6, 7, 8, 9, 10, 11, 12, 13, 14, 15, 16, 17, 18, 19, 20, 21, 22, 23, 24, or 25 nucleotides. In any of the embodiments herein, the 5′ hybridization region can comprise the interrogatory sequence and can be shorter than the 3′ hybridization region by about 5, 6, 7, 8, 9, 10, 11, 12, 13, 14, or 15 nucleotides. In any of the embodiments herein, the 5′ hybridization region can be about 1, 2, 3, 4, 5, 6, 7, 8, 9, 10, 11, 12, 13, 14, or 15 nucleotides shorter than the 3′ hybridization region.

In any of the embodiments herein, the target nucleic acid may be, e.g., an mRNA molecule. In any of the embodiments herein, the interrogatory region can comprise a single interrogatory nucleotide for detecting, e.g., a particular SNP in the target nucleic acid. In any of the embodiments herein, the interrogatory region can comprise more than one interrogatory nucleotide.

In any of the embodiments herein, the circularizable probe or probe set can comprise one, two, three, four, or more ribonucleotides. In some embodiments, a circularizable probe disclosed herein can comprise one, two, three, four, or more ribonucleotides in a DNA backbone. In any of the embodiments herein, the one or more ribonucleotides can be at and/or near a ligatable 3′ end of the circularizable probe or probe set. The circularizable probe may comprise an optional 3′ RNA base. The presence of a complementary base in the SNP location in the target nucleic acid favors stable hybridization of the circularizable probe and subsequent circularization. In contrast, the interrogatory nucleotide-containing arm is less stably hybridized when there is a mismatch between the interrogatory nucleotide and the SNP location in the target nucleic acid. The interrogatory nucleotide-containing arm can dissociate from the target nucleic acid and prevent circularization of the circularizable probe by a ligase having poor fidelity (e.g., on RNA templates), thus reducing false positive signals due to indiscriminating ligase activity.

In some embodiments, disclosed herein is a method for analyzing a biological sample, comprising contacting the biological sample with a circularizable probe, wherein the circularizable probe comprises a 5′ hybridization region and a 3′ hybridization region that hybridize to adjacent first and second target regions, respectively, in a target RNA in the biological sample, where the 5′ hybridization region and the 3′ hybridization region are different in lengths. In some embodiments, the shorter one of the 5′ hybridization region and the 3′ hybridization region comprises an interrogatory nucleotide complementary to a single nucleotide of interest in the first or second target region, respectively. For instance, the 5′ hybridization region can be the shorter one and can comprise the interrogatory nucleotide. In other examples, the 3′ hybridization region can be the shorter one and can comprise the interrogatory nucleotide. In any of the embodiments herein, the interrogatory nucleotide may not be a 5′ or 3′ terminal nucleotide.

In any of the embodiments herein, the interrogatory nucleotide can be an internal nucleotide in the 5′ hybridization region or the 3′ hybridization region. In any of the embodiments herein, the interrogatory nucleotide may not comprise a ligatable end. In any of the embodiments herein, the interrogatory nucleotide may not be a nucleotide which, with or without processing of the circularizable probe, participates in a subsequent ligation reaction, for example, a ligation that is catalyzed by a ligase having RNA-splinted ligase activity. In some embodiments, upon processing of the circularizable probe hybridized to the target nucleic acid, the interrogatory nucleotide may be exposed and participate in a subsequent ligation reaction catalyzed by a ligase having RNA-splinted ligase activity. In some embodiments, a ligatable 5′ phosphate group or 3′ hydroxyl group of the interrogatory nucleotide may be exposed upon processing of the circularizable probe. In any of the embodiments herein, the interrogatory nucleotide can be a ribonucleotide, a deoxyribonucleotide, or a variant or derivative thereof.

In some embodiments, a probe disclosed herein (e.g., a circularizable probe) can comprise a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiments, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in US 2020/022424, the content of which is herein incorporated by reference in its entirety, and may include P. furiosus (Pfu), A. fulgidus (Afu), M jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148 for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

In any of the embodiments herein, the interrogatory region can be in the hybridization region of the probe and not in the blocking strand. In some embodiments, while the blocking strand may comprise a region that is identical in sequence to the interrogatory region in the probe, the probe must comprise the interrogatory region such that the blocking strand and the probe can compete for hybridizing to the target nucleic acid in a sequence that includes the region of interest. In some embodiments, the region of interest is in the complementary hybridization region of the target nucleic acid, and the hybridization region hybridizes to the complementary hybridization region, e.g., by and/or following displacement of the blocking strand. In some embodiments, the blocking strand hybridizes to the interrogatory region in the hybridization region of the probe. The interrogatory region can be at the 5′ end, at the 3′ end, or between the 5′ end and the 3′ end of the hybridization region of the probe.

In some embodiments, the region of interest is in the hybridization region of the target nucleic acid, the interrogatory region is in the complementary hybridization region of the probe, and the hybridization region hybridizes to the complementary hybridization region. In some embodiments, the blocking strand hybridizes to the region of interest in the hybridization region of the target nucleic acid. The region of interest can be at the 5′ end, at the 3′ end, or between the 5′ end and the 3′ end of the hybridization region of the target nucleic acid.

In some embodiments, the interrogatory region is not in the blocking strand. In some embodiments, the interrogatory region for detecting the region of interest is in the probe and the blocking strand may but does not need to comprise any interrogatory region for detecting the region of interest. In some embodiments, the blocking strand does not comprise a contiguous sequence that is generated by the ligation of the probe to itself or to another probe, wherein the contiguous sequence spans a ligation point. In some embodiments, the blocking strand does not comprise a 5′ overhang and/or a 3′ overhang upon hybridization to the probe or to the target nucleic acid.

### IV. Ligation, Extension, and Amplification

In some aspects, after formation of a hybridization complex comprising nucleic acid probes and/or probe sets described in Section III and the target nucleic acids, the assay further comprises one or more steps such as ligation, extension and/or amplification of the probe or probe set hybridized to the target nucleic acid. In some embodiments, the methods of the invention include the step of performing rolling circle amplification in the presence of a target nucleic acid of interest.

In some embodiments, the method comprises using a circular or circularizable construct hybridized to the target nucleic acid comprising the region of interest to generate a product (e.g., comprising a sequence of the region of interest or one or more barcode sequences associated with the target nucleic acid). In some aspects, the product is generated using RCA. In any of the embodiments herein, the method can comprise ligating the ends of a circularizable probe hybridized to the target RNA to form a circularized probe. In any of the embodiments herein, the method can further comprise generating a rolling circle amplification product of the circularized probe. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In any of the embodiments herein, the method can further comprise detecting a signal associated with the rolling circle amplification product in the biological sample. In some embodiments, a ligation product of a first and second probe is generated. In some aspects, the ligation product or a derivative thereof (e.g., extension product) can be detected. In some cases, RCA is not performed.

In some embodiments, the circular construct is formed using ligation. In some embodiments, the circular construct is formed using template primer extension followed by ligation. In some embodiments, the circular construct is formed by providing an insert between ends to be ligated. In some embodiments, the circular construct is formed using a combination of any of the foregoing. In some embodiments, the ligation is a DNA-DNA templated ligation. In some embodiments, the ligation is an RNA-RNA templated ligation. In some embodiments, the ligation is a RNA-DNA templated ligation. In some embodiments, a splint is provided as a template for ligation.

In some embodiments, the circular construct is directly hybridized to the target nucleic acid. In some embodiments, the circular construct is formed from a circularizable probe. In some embodiments, the circular construct is formed from a probe or probe set capable of DNA-templated ligation. See, e.g., U.S. Pat. No. 8,551,710, which is hereby incorporated by reference in its entirety. In some embodiments, the circular construct is formed from a probe or probe set capable of RNA-templated ligation. Exemplary RNA-templated ligation probes and methods are described in US 2020/022424 which is incorporated herein by reference in its entirety. In some embodiments, the circular construct is formed from a specific amplification of nucleic acids via intramolecular ligation (e.g., SNAIL) probe set. See, e.g., U.S. Pat. Pub. 20190055594, which is hereby incorporated by reference in its entirety. In some embodiments, the circular construct is formed from a probe capable of proximity ligation, for instance a proximity ligation assay for RNA (e.g., PLAYR) probe set. See, e.g., U.S. Pat. Pub. 20160108458, which is hereby incorporated by reference in its entirety.

In some embodiments, the circular construct is indirectly hybridized to the target nucleic acid. In some embodiments, the circular construct is formed from a probe set capable of proximity ligation, for instance a proximity ligation in situ hybridization (PLISH) probe set.

The nature of the ligation reaction depends on the structural components of the polynucleotides used to form the circularizable or circular probe. In one embodiment, the polynucleotides comprise complementary docking regions that self-assemble the two or more polynucleotides into a circularizable probe that is either ready for ligation because no gaps exist between the docking regions, or is ready for a fill-in process, which will then permit the ligation of the polynucleotides to form the circularized probe. In another embodiment, the docking regions are complementary to a splint.

In some embodiments, a 3′ end and a 5′ end of the circularizable probe or probe set can be ligated using the target nucleic acid (e.g., RNA) as a template. In some embodiments, the 3′ end and the 5′ end are ligated without gap filling prior to ligation. In some embodiments, the ligation of the 3′ end and the 5′ end is preceded by gap filling. The gap may be 1, 2, 3, 4, 5, or more nucleotides.

In some embodiments, the circularizing step may comprise ligation selected from the group consisting of enzymatic ligation, chemical ligation, template dependent ligation, and/or template independent ligation. In any of the embodiments herein, the ligation can comprise using a ligase having an RNA-templated DNA ligase activity and/or an RNA-templated RNA ligase activity. In any of the embodiments herein, the ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any of the embodiments herein, the ligation can comprise using a PBCV-1 DNA ligase or variant or derivative thereof and/or a T4 RNA ligase 2 (T4 Rnl2) or variant or derivative thereof.

In some embodiments, the method can further comprise prior to the circularizing step, a step of removing molecules of the circularizable probe or probe set that are not bound to the target nucleic acid from the biological sample. In any of the embodiments herein, the method can further comprise prior to the circularizing step, a step of removing molecules of the circularizable probe or probe set that are bound to the target nucleic acid but comprise one or more mismatches in the interrogatory sequence from the biological sample. In any of the embodiments herein, the method can further comprise prior to the circularizing step, a step of allowing circularizable probe molecules that are bound to the target nucleic acid but comprise one or more mismatches in the interrogatory sequence to dissociate from the target nucleic acid, while circularizable probe molecules comprising no mismatch in the interrogatory sequence remain bound to the target nucleic acid. In any of the embodiments herein, under the same conditions, the molecules comprising one or more mismatches can be less stably bound to the target nucleic acid than the molecules comprising no mismatch in the interrogatory sequence. In any of the embodiments herein, the method can comprise one or more stringency washes. For instance, one or more stringency washes can be used to remove circularizable probe molecules that are not bound to the target nucleic acid, and/or circularizable probe molecules that are bound to the target nucleic acid but comprise one or more mismatches in the interrogatory sequence.

Following formation of a circularized probe, or otherwise providing a circular probe, in some instances, an amplification primer is added. In other instances, the amplification primer is added with the circularizable probes. In some instances, the amplification primer may also be complementary to the target nucleic acid and the circularizable probe (e.g., a SNAIL probe). In some embodiments, a washing step is performed to remove any unbound probes, primers, etc. In some embodiments, the wash is a stringency wash. Washing steps can be performed at any point during the process to remove non-specifically bound probes, probes that have ligated, etc. In some embodiments, the stringency is increased in the hybridization of the probe or probe set to the target nucleic acid, reducing or negating the need of performing a stringency wash.

Upon addition of a DNA polymerase in the presence of appropriate dNTP precursors and other cofactors, the amplification primer is elongated by replication of multiple copies of the template (e.g., a concatemer of the template is generated). This amplification product can be detected using, e.g., the secondary and higher order probes and detection oligonucleotides described herein. In some embodiments, the sequence of the amplicon or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, sequencing using, e.g., the secondary and higher order probes and detection oligonucleotides described herein.

In any of the embodiments herein, the method can further comprise generating the product of the circularized probe in situ in the biological sample. In any of the embodiments herein, the product can be generated using rolling circle amplification (RCA). In any of the embodiments herein, the RCA can comprise a linear RCA, a branched RCA, a dendritic RCA, or any combination thereof. In any of the embodiments herein, the product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In any of the embodiments herein, the product can be immobilized in the biological sample. In any of the embodiments herein, the product can be crosslinked to one or more other molecules in the biological sample.

In some embodiments, an exemplary workflow for analyzing a biological sample, comprises contacting the biological sample with a probe, wherein: the probe comprises a hybridization region that hybridizes to a target RNA in the biological sample; the hybridization region on the probe comprises an interrogatory region complementary to a region of interest in the target RNA, wherein a second molecule of the target RNA comprises a mismatch with the interrogatory nucleotide; and hybridization between the hybridization region on the probe and the region of interest on the target nucleic acid is blocked by a blocking strand that is hybridized to the hybridization region on the probe or the region of interest on the target nucleic acid. In some embodiments, the method further comprises allowing the probe or a portion thereof (e.g., the hybridization region) to dissociate from the second molecule of the target RNA, wherein under the same conditions, the circularizable probe (e.g., including the hybridization region) remains hybridized to the first molecule of the target RNA. In some embodiments, the method further comprises ligating the ends of the probe hybridized to the first molecule of the target RNA to form a circularized probe; generating a rolling circle amplification product of the circularized probe; and detecting a signal associated with the rolling circle amplification product in the biological sample. In any of the preceding embodiments, the probe hybridized to the second molecule of the target RNA can be destabilized and/or removed from the biological sample while the probe remains hybridized to the first molecule of the target RNA, such that a circularized probe hybridized to the second molecule of the target RNA is not formed. In any of the preceding embodiments, a signal associated with the second molecule of the target RNA may not be detected, such that a signal associated with the rolling circle amplification product is indicative of the first molecule and not the second molecule of the target RNA in the biological sample.

### V. Spatial Array Capture

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array. Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, US 2022/0010367, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

In some embodiments, a target nucleic acid such as an RNA or DNA in the biological sample can be transferred to an array. In some embodiments, a target nucleic acid in the biological sample can be migrated towards the array substrate and be captured by capture probes on the substrate. In some embodiments, a hybridization complex comprising probes and/or blocking strands described herein (e.g., in Section III) and the target nucleic acids can be formed on the array, e.g., by contacting the substrate with the captured target nucleic acids with the probes and/or blocking strands described herein. In some embodiments, after formation of a hybridization complex comprising probes and/or blocking strands described herein (e.g., in Section III) and the target nucleic acids in the biological sample, the assay may comprise one or more steps for transferring the probes (or a product or derivative thereof) to an array. In some embodiments, the probes (e.g., a first probe and a second probe) can be ligated (e.g., using RNA-templated ligation in the biological sample) and transferred to an array. In some embodiments, a product (e.g., extension product) or derivative of the ligated probes (e.g., ligated first-second probe) can be transferred to an array.

In some cases, spatial analysis can be performed by attaching and/or introducing a molecule having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. Some such methods of spatial analysis are described in Section (III) of US 2022/0010367 and/or U.S. Patent Application Publication No. 2020/0277663.

In some cases, spatial analysis can be performed by detecting multiple oligonucleotides (e.g., probe or probe set as described in Section III) that hybridize to a target nucleic acid (e.g., comprising a region of interest). In some instances, for example, spatial analysis can be performed using RNA-templated ligation (RTL). Methods of RTL have been described previously. See, e.g., US20180208967 incorporated herein by reference and Credle et al., Nucleic Acids Res. 2017 Aug. 21; 45(14):e128. Typically, RTL includes hybridization of two oligonucleotides (e.g., a first probe and a second probe) to adjacent sequences on an analyte (e.g., an RNA molecule, such as an mRNA molecule). In some instances, the oligonucleotides are DNA molecules. In some instances, one of the oligonucleotides (e.g., probes) includes at least two ribonucleic acid bases at the 3′ end and/or the other oligonucleotide includes a phosphorylated nucleotide at the 5′ end. In some instances, one of the two oligonucleotides (e.g., probes) includes a capture domain (e.g., a poly(A) sequence, a non-homopolymeric sequence). In some instances, one or both of the oligonucleotides (e.g., probes) may comprise an interrogatory region, and the interrogatory region(s) may be blocked by a blocking strand. The blocking strand may be part of either or both oligonucleotides (e.g., probes), or may be provided as a separate molecule. In some embodiments, the blocking strand is prehybridized to the one or both of the oligonucleotides prior to contacting the oligonucleotides with an analyte. In some embodiments, the blocking strand is prehybridized to a target nucleic acid in or associated with an analyte prior to contacting the target nucleic acid with one or both of the oligonucleotides. Sequence complementarity between an interrogatory region in one or both of the oligonucleotides and a sequence of interest (e.g., an SNP) in the target nucleic acid favors displacement of the blocking strand and stable hybridization of the two oligonucleotides to the target nucleic acid in or associated with the analyte. After hybridization to the analyte, a ligase (e.g., SplintR ligase) ligates the two oligonucleotides together, creating a ligation product. In some instances, the two oligonucleotides (e.g., probes) hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product (or a derivative thereof) can then be captured by capture probes (e.g., instead of direct capture of an analyte) on an array, optionally amplified, and sequenced, thus determining the location and optionally the abundance of the analyte in the biological sample.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of US 2022/0010367 and/or U.S. Patent Application Publication No. 2020/0277663. Generation of capture probes can be achieved by any appropriate method, including those described in Section (II)(d)(ii) of US 2022/0010367 and/or U.S. Patent Application Publication No. 2020/0277663.

In some embodiments, at least one of the probes for binding a target nucleic acid (e.g., probe or probe set as described in Section III) comprise a sequence complementary to a sequence comprised by a capture probe. In some embodiments, a plurality of the probes (e.g., first or second probes) comprise a common sequence for hybridizing to a capture probe. In some embodiments, the capture probes are spatially-barcoded capture probes attached to an array. In some examples, a ligation product (or a derivative thereof) can be captured by capture probes and associated with a spatial barcode, optionally amplified, and sequenced, thus determining the location of the target nucleic acid. In some cases, the spatially barcoded analyte (or a product or derivative thereof) can be released from the array prior to analysis. In some cases, the spatially barcoded analyte can be further processed and subjected to one or more reactions prior to analysis (e.g., extension, amplification, or other reactions described in Section IV).

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of US 2022/0010367 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of US 2022/0010367 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of US 2022/0010367 and/or U.S. Patent Application Publication No. 2020/0277663. Some quality control measures are described in Section (II)(h) of US 2022/0010367 and/or U.S. Patent Application Publication No. 2020/0277663.

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of US 2022/0010367 and/or U.S. Patent Application Publication No. 2020/0277663. Exemplary features and geometric attributes of an array can be found in Sections (II)(d)(i), (II)(d)(iii), and (II)(d)(iv) of US 2022/0010367 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of US 2022/0010367 and/or U.S. Patent Application Publication No. 2020/0277663.

During analysis of spatial information, sequence information for a spatial barcode associated with an analyte (e.g., ligated first-second probe or product or derivative thereof) is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of US 2022/0010367 and/or U.S. Patent Application Publication No. 2020/0277663. See, for example, the Exemplary embodiment starting with “In some non-limiting examples of the workflows described herein, the sample can be immersed . . . ” of US 2022/0010367 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of US 2022/0010367 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in US 2021/0150707.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in US 2021/0097684 and spatial analysis methods are generally described in US 2020/0089885 and/or US 2021/0155982.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of US 2022/0049294 and/or US 2021/0158522. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### VI. Detection and Analysis

In some aspects, after formation of a hybridization complex comprising nucleic acid probes and/or probe sets described in Section III and further processing (e.g., ligation, extension amplification, capture, or any combination thereof) as described in Section IV, the method further includes detection of the probe or probe set hybridized to the target nucleic acid or any products generated therefrom or a derivative thereof. In any of the embodiments herein, the method can further comprise imaging the biological sample to detect a ligation product or a circularized probe or product thereof. In any of the embodiments herein, a sequence of the ligation product, rolling circle amplification product, or other generated product can be analyzed in situ in the biological sample. In any of the embodiments herein, the imaging can comprise detecting a signal associated with a fluorescently labeled probe that directly or indirectly binds to a rolling circle amplification product of the circularized probe. In any of the embodiments herein, the sequence of the ligation product, rolling circle amplification product, or other generated product can be analyzed by sequential hybridization, sequencing by hybridization, sequencing by ligation, sequencing by synthesis, sequencing by binding, or a combination thereof. In some cases, a spatially barcoded analyte (or a product or derivative thereof) can be released from an array prior to analysis.

In any of the embodiments herein, a sequence associated with the target nucleic acid or the probe(s) can comprise one or more barcode sequences or complements thereof. In any of the embodiments herein, the sequence of the rolling circle amplification product can comprise one or more barcode sequences or complements thereof. In any of the embodiments herein, a ligated first-second probe can comprise one or more barcode sequences or complements thereof. In any of the embodiments herein, the one or more barcode sequences can comprise a barcode sequence corresponding to the target nucleic acid. In any of the embodiments herein, the one or more barcode sequences can comprise a barcode sequence corresponding to the sequence of interest, such as variant(s) of a single nucleotide of interest.

In any of the embodiments herein, the detecting step can comprise contacting the biological sample with one or more detectably-labeled probes that directly or indirectly hybridize to the rolling circle amplification product, and dehybridizing the one or more detectably-labeled probes from the rolling circle amplification product. In any of the embodiments herein, the contacting and dehybridizing steps can be repeated with the one or more detectably-labeled probes and/or one or more other detectably-labeled probes that directly or indirectly hybridize to the rolling circle amplification product.

In any of the embodiments herein, the detecting step can comprise contacting the biological sample with one or more intermediate probes that directly or indirectly hybridize to the rolling circle amplification product, wherein the one or more intermediate probes are detectable using one or more detectably-labeled probes. In any of the embodiments herein, the detecting step can further comprise dehybridizing the one or more intermediate probes and/or the one or more detectably-labeled probes from the rolling circle amplification product. In any of the embodiments herein, the contacting and dehybridizing steps can be repeated with the one or more intermediate probes, the one or more detectably-labeled probes, one or more other intermediate probes, and/or one or more other detectably-labeled probes.

In some embodiments, the detection may be spatial, e.g., in two or three dimensions. In some embodiments, the detection may be quantitative, e.g., the amount or concentration of a primary nucleic acid probe (and of a target nucleic acid) may be determined. In some embodiments, primary probes (e.g., probes that directly hybridize to a target nucleic acid), secondary probes (e.g., probes that directly hybridize to a primary probe, or a complex comprising the primary probe, or a product (e.g., RCA product) of the primary probe), higher order probes (e.g., probes that directly hybridize to a secondary probe, or a complex comprising the secondary probe, or a product (e.g., RCA product) of the secondary probe), and/or detectably labeled probes may comprise any of a variety of entities able to hybridize a nucleic acid, e.g., DNA, RNA, LNA, and/or PNA, etc., depending on the application.

In some embodiments, disclosed herein is a multiplexed assay where multiple targets (e.g., nucleic acids such as genes or RNA transcripts, or protein targets) are probed with multiple primary probes (e.g., circularizable primary probes), and optionally multiple secondary probes hybridizing to the primary barcodes (or complementary sequences thereof) are all hybridized at once, followed by sequential secondary barcode detection and decoding of the signals. In some embodiments, detection of barcodes or subsequences of the barcode can occur in a cyclic manner.

In some embodiments, a method for analyzing a region of interest in a target nucleic acid is a multiplexed assay where multiple probes (e.g., circularizable probes) are used to detect multiple regions of interest simultaneously (e.g., variations at the same location of a target nucleic acid and/or SNPs in various locations). In some embodiments, one or more detections of one or more regions of interest may occur simultaneously. In some embodiments, one or more detections of one or more regions of interest may occur sequentially. In some embodiments, multiple circularizable probes of the same circularizable probe design are used to detect one or more regions of interest, using different barcodes associated with each region of interest. In some embodiments, multiple circularizable probes of different circularizable probe design are used to detect one or more regions of interest, using different barcodes (e.g., each barcode associated with a target nucleic acid or sequence thereof). In some embodiments, the one or more regions of interest are localized on the same molecule (e.g., RNA or DNA). In alternative embodiments, the one or more single nucleotides of interest are localized on different molecules.

In some aspects, the provided methods involve analyzing, e.g., detecting or determining, one or more sequences present in the polynucleotides (e.g., probe or probe set) and/or in a product or derivative thereof, such as in an amplified circularizable probe. In some embodiments, the detection comprises providing detection probes, such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence present in a circularizable probe or product thereof).

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, the RCA product generated using a method disclosed herein can be detected in with a method that comprises signal amplification.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 the content of which is herein incorporated by reference in its entirety), hybridization chain reaction, assembly of topologically catenated DNA structures using serial rounds of chemical ligation (clampFISH), signal amplification via hairpin-mediated concatemerization (e.g., as described in US 2020/0362398 the content of which is herein incorporated by reference in its entirety), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, US 2022/0026433, US 2022/0128565, and US 2021/0222234, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product. Exemplary methods and compositions for LO-HCR are described in US 2021/0198723, incorporated herein by reference in its entirety.

In some embodiments, detection of nucleic acids sequences in situ includes combination of RCA with an assembly for branched signal amplification. In some embodiments, the assembly complex comprises an amplifier hybridized directly or indirectly (via one or more oligonucleotides) to a sequence of the RCA product. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, the RCA product can be detected in with a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising RCA products generated using methods described herein. In various embodiments, the RCA product may be contacted with a plurality of concatemer primers and a plurality of labeled probes. see e.g., U.S. Pat. Pub. No. US20190106733, which is incorporated herein by reference, for exemplary molecules and PER reaction components.

In some embodiments, the methods comprise sequencing all or a portion of the amplification product, such as one or more barcode sequences present in the amplification product. In some embodiments, extended capture probes can be amplified (e.g., in bulk solution or on the array) and analyzed, e.g., via DNA sequencing.

In some embodiments, the product or derivative of a first and second probe ligated together after hybridizing to the target nucleic acid can be analyzed by sequencing. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, the provided methods comprise imaging the amplification product (e.g., amplicon) and/or one or more portions of the polynucleotides, for example, via binding of the detection probe and detecting the detectable label. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. In some embodiments, the “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5- UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,192,782, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121). In some cases, sequencing can be performed after the analytes are released from the biological sample.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232.

In some embodiments, sequence analysis of nucleic acids (e.g., nucleic acids such as probes or RCA products comprising barcode sequences) can be performed by sequential hybridization (e.g., sequencing by hybridization and/or sequential in situ fluorescence hybridization). Sequential fluorescence hybridization can involve sequential hybridization of detectable probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and WO 2021/138676, all of which are incorporated herein by reference. In some embodiments, the methods provided herein can include analyzing the identifier sequences (e.g., analyte sequences or barcode sequences (by sequential hybridization and detection with a plurality of labeled probes (e.g., detection oligonucleotides).

In some embodiments, sequence detection comprises contacting the biological sample with one or more intermediate probes that directly or indirectly hybridize to a rolling circle amplification product, wherein the one or more intermediate probes are detectable using one or more detectably-labeled probes, and dehybridizing the one or more intermediate probes and/or the one or more detectably-labeled probes from the rolling circle amplification product. In some embodiments, the one or more intermediate probes comprise one or more overhang regions (e.g., a 5′ and/or 3′ end of the probe that does not hybridize to the rolling circle amplification product). A probe comprising a single overhang region may be referred to as an “L-shaped probe,” and a probe comprising two overhangs may be referred to as a “U-shaped probe.” In some cases, the overhang region comprises a binding region for binding one or more detectably-labeled probes. In some embodiments, the detecting comprises contacting the biological sample with a pool of intermediate probes corresponding to different barcode sequences or portions thereof, and a pool of detectably-labeled probes corresponding to different detectable labels. In some embodiments, the biological sample is sequentially contacted with different pools of intermediate probes. In some instances, a common or universal pool of detectably-labeled probes is used in a plurality of sequential hybridization steps (e.g., with different pools of intermediate probes).

In some embodiments, provided herein are methods for in situ analysis of analytes in a sample using sequential probe hybridization. In some aspects provided herein is a method for analyzing a biological sample, comprising: a) generating a rolling circle amplification product (RCP) of a circularizable probe or probe set described herein, the RCP comprising an identifier sequence such as a barcode sequence or analyte sequence, wherein the identifier sequence is associated with an analyte of interest and is assigned a signal code sequence; b) contacting the biological sample with a first probe (e.g., an intermediate probe such as an L-probe) and a first detectably labeled probe to generate a first complex comprising the first probe hybridized to the RCP and the first detectably labeled probe hybridized to the first probe, wherein the first probe comprises (i) a recognition sequence (e.g., a target-binding sequence) complementary to the identifier sequence (e.g., barcode sequence or analyte sequence) and (ii) a first landing sequence (e.g., an overhang sequence), and wherein the first detectably labeled probe comprises a sequence complementary to the first landing sequence; c) detecting a first signal associated with the first detectably labeled probe, wherein the first signal corresponds to a first signal code in the signal code sequence; d) contacting the biological sample with a second probe (e.g., an intermediate probe such as L-probe) and a second detectably labeled probe to generate a second complex comprising the second probe hybridized to the RCP and the second detectably labeled probe hybridized to the second probe, wherein the second probe comprises (i) a recognition sequence (e.g., a target-binding sequence) complementary to the identifier sequence (e.g., barcode sequence or analyte sequence) and (ii) a second landing sequence (e.g., an overhang sequence), and wherein the second detectably labeled probe comprises a sequence complementary to the second landing sequence; and e) detecting a second signal associated with the second detectably labeled probe, wherein the second signal corresponds to a second signal code in the signal code sequence, wherein the signal code sequence comprising the first signal code and the second signal code is determined at a location in the biological sample, thereby decoding the identifier sequence (e.g., barcode sequence or analyte sequence) and identifying the analyte of interest at the location in the biological sample. In some embodiments, the detectable label of the first detectably labeled probe and the detectable label of the second detectably labeled probe are the same. In some embodiments, the detectable labels of the first detectably labeled probe and the second detectably labeled probe are different. In some embodiments, the first signal code and the second signal code are the same. In some embodiments, the first signal code and the second signal code are different.

In some embodiments, the first probe (e.g., a first intermediate probe such as a first L-probe), the second probe (e.g., a second intermediate probe such as a second L-probe), and one or more subsequent probes (e.g., subsequent intermediate probe such as subsequent L-probes) are contacted with the biological sample sequentially in a pre-determined sequence which corresponds to the signal code sequence assigned to the identifier sequence (e.g., barcode sequence or analyte sequence), wherein the one or more subsequent probes each comprises (i) a recognition sequence complementary to the identifier sequence (e.g., barcode sequence or analyte sequence) and (ii) an overhang sequence complementary to a detectably labeled probe of a pool (e.g., a universal pool across different cycles of probe hybridization) of detectably labeled probes. In some embodiments, the biological sample is contacted with the first probe before the second probe and one or more subsequent probes. In some embodiments, the biological sample is contacted with the second after the first probe and before and one or more subsequent probes. In some embodiments, the biological sample is contacted with the one or more subsequent probes after the first probe. In some embodiments, the biological sample is contacted with the one or more subsequent probes after the first probe and the second probe.

In some embodiments, the first detectably labeled probe and the second detectably labeled probe are in the pool of detectably labeled probes. A pool of detectably labeled probes may comprises at least two detectably labeled probes, and may be used for multiplexing analyses of two or more target analytes (e.g., target nucleic acids) in a biological sample. In some embodiments, the contacting in b) comprises contacting the biological sample with the universal pool of detectably labeled probes, and the contacting in d) comprises contacting the biological sample with the universal pool of detectably labeled probes. In some embodiments, the universal pool of detectably labeled probes used in the contacting in b) is the same as the universal pool of detectably labeled probes used in the contacting in d). In some embodiments, the universal pool comprises detectably labeled probes each having a detectable label corresponding to a different nucleic acid sequence for hybridization to a landing sequence (e.g., an overhang sequence) in a probe (e.g., an intermediate probe such as an L-probe). In some embodiments, the number of different detectably labeled probes in the universal pool is four.

In some embodiments, the one or more subsequent probes are contacted with the biological sample to determine signal codes in the signal code sequence until sufficient signal codes have been determined to decode the identifier sequence (e.g., barcode sequence or analyte sequence), thereby identifying the target analyte (e.g., target nucleic acid). In some embodiments, the method further comprises a step of removing the first probe and/or the first detectably labeled probe from the biological sample before contacting the sample with a subsequent probe and a detectably labeled probe hybridizing to the subsequent probe. In some embodiments, the method further comprises a step of removing the second probe and/or the second detectably labeled probe from the biological sample, before contacting the sample with a subsequent probe and a detectably labeled probe hybridizing to the subsequent probe.

In some embodiments, the method further comprises identifying multiple different target analytes present at locations (e.g., different locations) in the biological sample. In some embodiments, each different target analyte is assigned a different signal code sequence and is targeted by a circularizable probe or probe set comprising a complement of a different barcode sequence of the plurality of barcode sequences. In some embodiments, the number of different probes (e.g., L-probes that have different recognition sequences that bind to the barcode sequences) in each pool of probes is greater than the number of different detectably labeled probes in the universal pool of detectably labeled probes. In some embodiments, the number of different detectably labeled probes in the universal pool is four. In some embodiments, the number of different probes in each pool of probes (e.g., L-probes) is about 10, about 20, about 30, about 40, about 50, about 100, about 200, about 500, about 1,000, or more. In some embodiments, the number of different recognition sequences (e.g., recognition sequences that bind to the barcode sequences) of probes in each pool of probes in at least about 10, such as at least any of about 20, 30, 40, 50, 100, 200, 500, 1,000, or more.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; US and U.S. Pat. No. 6,306,597.

In some embodiments, the barcodes of the probes (e.g., the circularizable probe or a spatially barcoded analyte comprising a sequence of the ligated probes) or complements or products thereof are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):e112; U.S. Pat. No. 10,457,980 B2; US 2016/0369329 A1; US 2021/0017587; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VII. Compositions, Kits, and Systems

In some embodiments, disclosed herein is a composition that comprises a complex containing a target nucleic acid, a probe, e.g., any of the probes described herein, and/or a blocking strand. In some embodiments, the complex further comprises a primer, e.g., for rolling circle amplification of a circularized padlock probe.

In some embodiments, disclosed herein is a composition that comprises an amplification product containing monomeric units of a sequence complementary to a sequence of a probe (e.g., a circularized probe). In some embodiments, the amplification product is formed using any of the target nucleic acids, probes (e.g., circularizable probes) and any of the amplification techniques described herein.

Also provided herein are kits, for example comprising one or more polynucleotides, e.g., any described in Section III, and reagents for performing the methods provided herein, for example reagents required for one or more steps comprising hybridization, ligation, amplification, detection, sequencing, and/or sample preparation as described herein. In some embodiments, the kit further comprises a target nucleic acid. In some embodiments, any or all of the polynucleotides are DNA molecules. In some embodiments, the target nucleic acid is a messenger RNA molecule. In some embodiments, the blocking strand is pre-hybridized to the probe. In some embodiments, the blocking strand is pre-hybridized to the target nucleic acid. In some embodiments, separate blocking strands are separately pre-hybridized to the probe and the target nucleic acid, respectively. In some embodiments, the kit further comprises a ligase, for instance for forming a ligated circular probe from the padlock probe. In some embodiments, the ligase has DNA-splinted DNA ligase activity. In some embodiments, the kit further comprises a polymerase, for instance for performing amplification of the padlock probe. In some embodiments, the polymerase is capable of using the ligated circular probe as a template for amplification. In some embodiments, the kit further comprises a primer for amplification. In some embodiments, the kits may further comprise one or more reagents for array based analysis, such as capture probes, e.g., any described in Section V.

Disclosed herein in some aspects is a kit for detecting a region of interest in a target nucleic acid, comprising: i) the target nucleic acid comprising a hybridization region comprising the region of interest, ii) a probe comprising a hybridization region comprising an interrogatory region, and/or iii) a blocking strand, wherein: the hybridization regions in the target nucleic acid and the probe are capable of hybridizing to each other, the blocking strand comprises a blocking sequence complementary to the interrogatory region or to the region of interest, the blocking sequence is optionally pre-hybridized to the hybridization region in the probe or the target nucleic acid, thereby blocking the hybridization regions in the target nucleic acid and the probe from hybridizing to each other, and the probe or the target nucleic acid comprises a toehold region adjacent to the interrogatory region or the region of interest, respectively.

In some aspects, disclosed herein is a kit for analyzing a biological sample comprising a plurality of target RNA molecules comprising a single nucleotide of interest, comprising: a circularizable probe hybridized to a blocking strand, wherein: the blocking strand is hybridized to a hybridization region in the circularizable probe, thereby blocking the hybridization region from hybridizing to a hybridization region in a target RNA molecule, the hybridization region in the circularizable probe comprises an interrogatory nucleotide hybridized to a blocking nucleotide in the blocking strand, and the circularizable probe comprises a toehold region directly linked to the interrogatory nucleotide via a phosphodiester bond.

In some aspects, disclosed herein is a kit for analyzing a biological sample, comprising: i) a target nucleic acid in the biological sample, ii) a probe, and/or iii) a blocking strand, wherein: the blocking strand is hybridized to a hybridization region in the probe or the target nucleic acid, thereby blocking the hybridization region from hybridizing to a hybridization region in the target nucleic acid or the probe, respectively, and the probe or the target nucleic acid comprises a toehold region adjacent to the hybridization region.

In some aspects, disclosed herein is a kit for detecting a region of interest in a target nucleic acid, comprising: the target nucleic acid comprising the region of interest and/or a circularizable probe comprising a metastable stem-loop structure, wherein: a strand of the stem of the metastable stem-loop structure comprises an interrogatory region, and the circularizable probe further comprises a toehold region adjacent to the interrogatory region.

In some aspects, disclosed herein is a kit for analyzing a biological sample comprising a plurality of target RNA molecules comprising a single nucleotide of interest, the kit comprising: a circularizable probe comprising a metastable stem-loop structure, wherein: a strand of the stem of the metastable stem-loop structure comprises an interrogatory nucleotide, and the circularizable probe further comprises a toehold region adjacent to the interrogatory nucleotide.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VIII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in a spatial array. In some aspects, the embodiments can be applied in an imaging or detection method for multiplexed nucleic acid analysis. In some aspects, the provided embodiments can be used to identify or detect regions of interest in target nucleic acids.

In some embodiments, the region of interest comprises more than one nucleotide of interest. In some embodiments, the region of interest is a single nucleotide of interest. In some embodiments, the single nucleotide of interest is a single-nucleotide polymorphism (SNP). In some embodiments, the single nucleotide of interest is a single-nucleotide variant (SNV). In some embodiments, the single nucleotide of interest is a single-nucleotide substitution. In some embodiments, the single nucleotide of interest is a point mutation. In some embodiments, the single nucleotide of interest is a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### IX. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.”

A “hybridization complex” as used herein may comprise one, two, or more strands or separate molecules. A hybridization complex that comprises three or more strands or separate molecules does not necessarily comprise direct hybridization between every possible pairwise combination thereof, so long as at least two molecules or strands are directly hybridized to each other, or are in the process of binding to or unbinding from each other, at a given time.

“Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1× SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“SNP” or “single nucleotide polymorphism” may include a genetic variation between individuals; e.g., a single nitrogenous base position in the DNA of organisms that is variable. SNPs are found across the genome; much of the genetic variation between individuals is due to variation at SNP loci, and often this genetic variation results in phenotypic variation between individuals. SNPs for use in the present invention and their respective alleles may be derived from any number of sources, such as public databases (U.C. Santa Cruz Human Genome Browser Gateway (genome.ucsc.edu/cgi-bin/hgGateway) or the NCBI dbSNP web site (www.ncbi.nlm.nih gov/SNP/), or may be experimentally determined as described in U.S. Pat. No. 6,969,589; and US Pub. No. 2006/0188875 entitled “Human Genomic Polymorphisms.” Although the use of SNPs is described in some of the embodiments presented herein, it will be understood that other biallelic or multi-allelic genetic markers may also be used. A biallelic genetic marker is one that has two polymorphic forms, or alleles. As mentioned above, for a biallelic genetic marker that is associated with a trait, the allele that is more abundant in the genetic composition of a case group as compared to a control group is termed the “associated allele,” and the other allele may be referred to as the “unassociated allele.” Thus, for each biallelic polymorphism that is associated with a given trait (e.g., a disease or drug response), there is a corresponding associated allele. Other biallelic polymorphisms that may be used with the methods presented herein include, but are not limited to multinucleotide changes, insertions, deletions, and translocations. It will be further appreciated that references to DNA herein may include genomic DNA, mitochondrial DNA, episomal DNA, and/or derivatives of DNA such as amplicons, RNA transcripts, cDNA, DNA analogs, etc. The polymorphic loci that are screened in an association study may be in a diploid or a haploid state and, ideally, would be from sites across the genome.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “adjacent” as used herein includes but is not limited to being directly linked by a phosphodiester bond. For example, “adjacent” nucleotides or regions on a nucleic acid such as a probe may be separated by a number of nucleotides. For instance, a toehold region and an interrogatory region adjacent to each other may be separated by 0, 1, 2, 3, 4, 5, 6, 7, 8, 9, 10 or more nucleotides in a probe.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

The term “each,” when used in reference to a collection of items, is intended to identify an individual item in the collection but does not necessarily refer to every item in the collection, unless expressly stated otherwise, or unless the context of the usage clearly indicates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

### Example 1: Use of a Metastable Stem-Loop Padlock Probe to Increase Specificity and Stringency When Detecting a Region of Interest in a Target mRNA Molecule

This example describes the design and use of a modified padlock probe with an internal metastable stem-loop structure for detecting a region of interest in a target nucleic acid, such as a SNP in messenger RNA.

In some examples shown in FIGS. 3-4, the probe is a modified standard circularizable padlock probe, wherein either arm of the padlock probe comprises an internal metastable stem-loop structure. Such an approach can be used to increase specificity and stringency when detecting a region of interest in a target nucleic acid, such as an mRNA. The stem strands of the metastable stem-loop structure are complementary to and hybridized to each other. The strand portion of the metastable stem-loop structure comprises an interrogatory region. The strands are connected by the loop portion of the metastable stem-loop structure. Adjacent to the interrogatory region is a toehold region. The toehold region may serve as the initial binding region of the padlock arm to the target nucleic acid when the probe and target come into contact. Thus, the location of the first part of the stem portion to hybridize to the target is determined by the relative location of the toehold region in a given probe. As shown in FIG. 3, the toehold region may be “below” the stem-loop structure towards either end (5′ or 3′) of the probe, or may be within the stem-loop structure itself, on the loop portion between the stems. In some embodiments, the toehold region can be adjacent to the stem-loop structure in an internal region of the probe (e.g., the stem-loop structure may be between the toehold region and an end of the padlock probe). As an example of the former situation, if the stem-loop structure is near one end of the probe, similar to the probe shown in FIG. 4, the toehold may be located in, e.g., a 5 bp overhang near the ligation site of the probe. In some embodiments, a loop of the metastable stem-loop structure of a probe may act as the toehold region, wherein hybridization of the loop or portion thereof to the target nucleic acid can initiate strand displacement and allow the interrogatory region to bind the region of interest of the target nucleic acid.

The sequence of the stem-loop structure can be designed for each individual target nucleic acid, as the interrogatory region in the stem is capable of both hybridizing to the other strand of the stem in order to form the metastable stem-loop structure, and hybridizing to the region of interest in the target when the stem-loop structure is unfolded. As such, the ideal stem and loop lengths for a given target may depend on the target sequence. Thus, the exact sequence length of the stem and the loop portions of the stem-loop structure may vary. A given stem-loop structure may comprise, for example, a 5 bp stem and a 15 bp loop, but there is no strict limit or range for the lengths of the stem and loop per se, so long as the probe is capable of forming a metastable stem-loop sequence that unfolds when contacted with a complementary region of interest in a target sequence that is available for hybridizing.

A probe comprising a metastable stem-loop structure such as those depicted in FIG. 3 could be used to increase specificity and stringency by only allowing the hairpin to bind if the full sequence is present on the mRNA molecule. Such an increase in specificity and stringency may negate the need of a stringency wash, simplifying the procedure. This is an improvement from the standard chimeric padlock design, which, while specific, requires a stringency wash to prevent non-specific binding padlocks from generating signal.

Currently available chimeric padlock probes are not fully capable of specifically detecting SNPs, potentially due to base bias regarding the 3′ terminal RNA base. By instead placing the interrogatory base, which is complementary to the SNP in the target mRNA, within the stem of the metastable stem-loop structure, the padlock arm will not properly bind unless the complementary SNP is present.

A probe set mixture is incubated with a thin tissue section sample and hybridization buffer for hybridization of the probe sets to target nucleic acid (e.g., mRNAs) in the sample. As shown in FIG. 3, if the complementary SNP is not present, the padlock probe would still bind the mRNA target with, for example, the 3′ arm and the toehold region on the 5′ arm. However, the metastable stem-loop structure will only open and allow the binding of the full padlock arm if the correct, complementary SNP is present. The presence of the complementary base to the SNP within the stem of the stem-loop structure would allow the slow opening and destabilization of the stem-loop, as the hybridization process is initiated by the toehold region. If the SNP is not present on the mRNA, the complementary base in the stem will not hybridize to the mRNA, and the hybridization process is halted, such that the stem-loop structure is not destabilized. Because the arm is not fully hybridized, the padlock would be removed in a subsequent stringency wash. If the interrogatory region is complementary to the region of interest in the target mRNA, then stem-loop structure will destabilize in favor of hybridizing to the target mRNA, such that the padlock probe will be stably hybridized to the target. In a padlock-type probe such as the one exemplified in FIGS. 3-4, the probe is designed such that stable hybridization of the probe to the sequence of interest results in physical proximity of the two termini of the padlock probe. For instance, the other arm of the probe may be hybridized to an adjacent region on the target nucleic acid. Such probes can then be ligated, amplified, and detected as outlined in, e.g., Section IV above.

The sample is then washed and incubated at room temperature with a T4 DNA ligase for ligation of the 5′ and 3′ ends of the padlock probes to form circular probes. A primer for amplification of the circular probe (e.g., detection padlock) may be added. The sample is then incubated with a rolling-circle amplification (RCA) mixture containing a Phi29 DNA polymerase and dNTP for RCA of the circular probes. Fluorescently labeled oligonucleotides complementary to a portion of the RCA product, a barcode contained therein, or a secondary probe attached thereto are incubated with the sample. Multiple cycles of contacting the sample with probes and sequence determination (e.g., using in situ sequencing based on sequencing-by-ligation or sequencing-by-hybridization) can be performed. Fluorescent images can be obtained in each cycle, and one or more wash steps can be performed in a cycle or between cycles. Probe targeting various SNPs within or across genes can be sequentially or simultaneously provided, processed, and detected as described above.

### Example 2: Use of a Padlock Probe and Separate Blocking Strand to Increase Specificity and Stringency When Detecting a Region of Interest in a Target mRNA Molecule

This example describes the design and use of a circularizable padlock probe and a separate blocking strand for detecting a region of interest in a target nucleic acid, such as a SNP in messenger RNA.

In some examples shown in FIGS. 1-2, the probe is a modified standard chimeric padlock probe for detecting an SNP, except that unlike in a standard chimeric padlock probe, the interrogatory base is shifted internally to, e.g., the 6th position inwards on the probe arm from the ligation site, and a second, shorter (e.g., 15 bp) polynucleotide (“blocking strand”) that is complementary to the probe arm is hybridized to the arm prior to hybridization of the padlock to the mRNA. This concept is similar to the stem-loop approach exemplified above in Example 1, except that instead of being covalently attached to the probe via a loop structure, the blocking strand here is on a different and separate molecule from the probe. This mechanism provides a method that similarly increases specificity and stringency but without the need to design a metastable stem-loop structure into the probe sequence.

Much like the stem portions of a metastable stem-loop probe such as those exemplified above in Example 1, the blocking strand here comprises a blocking sequence that is hybridized to a hybridization region on the probe, which prevents the hybridization region of the probe from stably hybridizing to the sequence of interest on the target nucleic acid unless the sequence of interest on the target is complementary to the hybridization region of the probe.

The hybridization region on the probe comprises an interrogatory region, which may be at any position or positions in the hybridization region. In FIG. 2, for example, the interrogatory region comprises a single guanine (G) nucleotide at the position that is complementary to both the 5′ terminal nucleotide of the blocking strand and the SNP of interest on the target. The probe also comprises a toehold region, which is adjacent to the hybridization region and does not hybridize to the blocking strand. In FIGS. 1-2, for example, the toehold region comprises a roughly 5 bp overhang between the hybridization region and the ligation site on the 5′ padlock arm. The toehold region recognizes the target nucleotide sequence (in this Example, an mRNA sequence) and initializes binding of the probe thereto. Binding of the toehold to the target initiates toehold-mediated displacement of the blocking strand, during which branch migration (hybridization of the target nucleic acid to the probe and de-hybridization of the blocking strand from the probe) takes place in the direction of the toehold region towards the hybridization region, as shown in FIG. 2.

A probe set comprising the probe and the blocking strands mixture is incubated with a thin tissue section sample and hybridization buffer for hybridization of the probe sets to target nucleic acid (e.g., mRNAs) in the sample. The blocking strand will only be fully displaced if the base or bases comprising the region of interest in the target nucleic acid are complementary to the interrogatory region of the probe. If the interrogatory region is complementary to a terminus of the blocking strand and immediately adjacent to the toehold region, as in FIGS. 1-2, displacement of this first base will be the limiting factor in displacement of the blocking strand. If the sequence of interest in the target nucleic acid, such as the SNP on the mRNAs exemplified in FIGS. 1-2, matches the interrogatory region on the probe, then displacement of the blocking strand continues efficiently. If, on the other hand, the regions are not complementary at every position, then the efficiency of the displacement of the blocking strand will be severely reduced and possibly interrupted. Incompletely hybridized probes, such as those without full blocking strand displacement, can be removed with a stringent wash.

Alternatively to hybridizing to the probe, the blocking strand in this approach may instead be designed to hybridize to the target nucleic acid. In such instances, the blocking strand will be pre-hybridized to the target rather than the probe. The principle remains the same, however: the blocking strand blocks hybridization between the probe and the sequence of interest unless the interrogatory region is complementary to the sequence of interest. This same principle could also be applied using a series of blocking strands, which each hybridize to different regions; for instance, one or more blocking strands may hybridize to various regions of the probe, and one or more additional blocking strands may hybridize to various regions of the sequence of interest, such that the hybridization region(s) on the probe would be unavailable for binding to the target unless the sequence(s) of interest on the target are complementary to the interrogatory region(s) of the probe. After hybridization, ligation, amplification and detection of the bound probes can be performed as described in Example 1. Probe sets including the blocking strands and padlock probes targeting various SNPs within or across genes can be sequentially or simultaneously provided, processed, and detected as described above.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method for detecting a region of interest in a target nucleic acid, the method comprising:
a) providing i) a probe comprising an interrogatory region and ii) a blocking strand, wherein:
the blocking strand is hybridized to a hybridization region in the probe or the target nucleic acid, thereby blocking the hybridization region from hybridizing to a complementary hybridization region in the target nucleic acid or the probe, respectively, and
the blocking strand comprises a blocking sequence complementary to the interrogatory region in the probe or the region of interest in the target nucleic acid;
b) allowing hybridization between the probe and the target nucleic acid, wherein if the interrogatory region is complementary to the region of interest, the blocking strand is displaced and the hybridization region is available for hybridizing to the target nucleic acid or the probe;
c) ligating the probe hybridized to the target nucleic acid to itself or to another probe hybridized to the target nucleic acid; and
d) detecting the ligated probe or an amplification product thereof, thereby detecting the region of interest in the target nucleic acid.

2-3. (canceled)

4. The method of claim 1, wherein the blocking strand and the probe are in different molecules.

5. (canceled)

6. The method of claim 1, wherein the blocking strand and the probe are in the same molecule, wherein:
the probe is a circularizable probe comprising a metastable stem-loop structure,
a strand of the stem of the metastable stem-loop structure comprises the interrogatory region, and the other strand of the stem is the blocking strand, and
if the interrogatory region is complementary to the region of interest, the blocking strand is displaced and the strand comprising the interrogatory region is available for hybridizing to the target nucleic acid.

7-8. (canceled)

9. The method of claim 1, wherein the probe is a circularizable probe and the ligating in step c) circularizes the circularizable probe.

10-11. (canceled)

12. The method of claim 9, comprising generating a rolling circle amplification product of the circularized circularizable probe and detecting the rolling circle amplification product.

13-21. (canceled)

22. The method of claim 1, wherein the probe comprises a ribonucleotide at its 3′ end.

23. The method of claim 1, wherein the interrogatory region is not at the 3′ or 5′ end of the probe.

24. (canceled)

25. The method of claim 1, wherein the blocking sequence is at the 3′ or 5′ end of the blocking strand.

26-27. (canceled)

28. The method of claim 1, wherein each of the interrogatory region, the blocking sequence, and the region of interest is a single nucleotide.

29. (canceled)

30. The method of claim 1, wherein the interrogatory region or the region of interest is at the 5′ end, at the 3′ end, or between the 5′ end and the 3′ end of the hybridization region.

31. The method of claim 1, wherein the probe or the target nucleic acid comprises a toehold region adjacent to the interrogatory region or the region of interest, respectively, and wherein in the hybridization of step b), the toehold region hybridizes to the target nucleic acid or the probe, thereby allowing displacement of the blocking strand.

32-39. (canceled)

40. The method of claim 1, further comprising prior to the ligating step, a step of removing probe molecules that are bound to the target nucleic acid but comprise in the interrogatory region one or more mismatches with the region of interest, and/or allowing probe molecules or portions thereof comprising one or more mismatches to dissociate from the target nucleic acid while probe molecules comprising no mismatch in the interrogatory region remain bound to the target nucleic acid.

41. The method of claim 40, wherein under the same conditions, probe molecules comprising one or more mismatches are less stably bound to the target nucleic acid than probe molecules comprising no mismatch in the interrogatory region.

42. The method of claim 40, wherein the removing step and/or the allowing step comprise one or more stringency washes.

43. The method of claim 1, wherein the target nucleic acid is in a biological sample and the ligated probe and/or the amplification product thereof is a rolling circle amplification (RCA) product generated in situ in the biological sample.

44-51. (canceled)

52. The method of claim 43, wherein the ligated probe and/or the amplification product thereof comprise one or more barcode sequences or complements thereof.

53. (canceled)

54. The method of claim 52, wherein the one or more barcode sequences or complements thereof are detected by:
contacting the biological sample with one or more detectably-labeled probes that directly or indirectly hybridize to the one or more barcode sequences or complements thereof,
detecting signals associated with the one or more detectably-labeled probes, and
dehybridizing the one or more detectably-labeled probes.

55. (canceled)

56. A method for analyzing a biological sample comprising a plurality of target RNA molecules comprising a single nucleotide of interest, the method comprising:
a) contacting the biological sample with a circularizable probe hybridized to a blocking strand, wherein:
the blocking strand is hybridized to a hybridization region in the circularizable probe, thereby blocking the hybridization region from hybridizing to a complementary hybridization region in a target RNA molecule,
the hybridization region in the circularizable probe comprises an interrogatory nucleotide hybridized to a blocking nucleotide in the blocking strand, and
the circularizable probe comprises a toehold region directly linked to the interrogatory nucleotide via a phosphodiester bond;
b) allowing hybridization of the toehold region to target RNA molecules in the biological sample, wherein:
for a first target RNA molecule in which the single nucleotide of interest is complementary to the interrogatory nucleotide, the blocking strand is displaced and the hybridization region of the circularizable probe is available for hybridizing to the first target RNA molecule, and
for a second target RNA molecule in which the single nucleotide of interest is not complementary to the interrogatory nucleotide, the blocking strand is not displaced and the hybridization region of the circularizable probe remains unavailable for hybridizing to the second target RNA molecule;
c) allowing molecule(s) of the circularizable probe to dissociate from the second target RNA molecule, under conditions in which molecule(s) of the circularizable probe remain hybridized to the first target RNA molecule;
d) circularizing the circularizable probe hybridized to the first target RNA molecule; and
e) detecting a rolling circle amplification product of the circularized circularizable probe in the biological sample.

57-85. (canceled)

86. The method of claim 1, wherein the hybridization region is in the probe, and the blocking strand hybridizes to the hybridization region and blocks it from hybridizing to the target nucleic acid.

87. The method of claim 1, wherein the hybridization region is in the target nucleic acid, and the blocking strand hybridizes to the hybridization region and blocks it from hybridizing to the probe.
